(19) United States
(12) Patent Application Publication
(10) Pub. No.: US 2010/0137693 A1
(43) Pub. Date: Jun. 3, 2010

(54) METHODS AND SYSTEMS FOR PATIENT CARE

(75) Inventors: Alexis Porras, Lexington, MA (US); Ravi Kalathil, Waltham, MA (US); Darrell Kauthen, Littleton, CO (US); Theresa J. Hetzel, Somerville, MA (US)

Correspondence Address: NUTTER MCCLENNEN & FISH LLP, SEAPORT WEST, 155 SEAPORT BOULEVARD, BOSTON, MA, 02210-2604, US

(73) Assignee: FRESENIUS MEDICAL CARE HOLDINGS, INC., Waltham, MA (US)

(21) Appl. No.: 12/620,283

(22) Filed: Nov. 17, 2009

Related U.S. Application Data
(63) Continuation-in-part of application No. 12/233,126, filed on Sep. 18, 2008.
Application No. 12/233,126 is a continuation-in-part of application No. 11/591,438, filed on Nov. 1, 2006.
(60) Provisional application No. 61/199,470, filed on Nov. 17, 2008.
(60) Provisional application No. 60/973,676, filed on Sep. 19, 2007.
(60) Provisional application No. 60/732,335, filed on Nov. 1, 2005.

(51) Int. Cl. A61B 5/00; G06Q 50/00

(52) U.S. Cl. 600/301; 705/2; 705/3

(57) ABSTRACT

Methods and apparatus for patient care detect a current medical condition of a patient receiving medical treatment, e.g., by sensing blood pressure, heart rate, weight, glucose level, hemoglobin level, and/or blood potassium level (all by way of example) and transmit information regarding that medical condition to a digital data processing system disposed remotely from the medical treatment apparatus. The medical treatment apparatus can be, for example, one for peritoneal dialysis and hemodialysis, and the medical condition can be sensed by sensors coupled to or otherwise utilized in connection with such apparatus.

This application claims the benefit of priority of U.S. Provisional Patent Application 61/199,470, filed Nov. 17, 2008, entitled “Patient-Specific Content Delivery Methods and Systems,” the teachings of which are incorporated herein by reference.

This application is also a continuation-in-part of U.S. patent application Ser. No. 12/233,126, filed Sep. 18, 2008, which claims the priority of U.S. Patent Application Ser. No. 60/973,676, filed Sep. 19, 2007, the teachings of both of which are incorporated herein by reference.

This application is also a continuation-in-part and claims the benefit of priority of U.S. patent application Ser. No. 11/591,438, filed Nov. 1, 2006, which claims the priority of U.S. Patent Application Ser. No. 60/732,335, filed Nov. 1, 2005, the teachings of both of which are incorporated herein by reference.

### BACKGROUND OF THE INVENTION

Dialysis is an important treatment regimen for a variety of chronic diseases. To meet the need for regular care, patients typically travel to hospitals or dialysis centers that are designed for efficient and routine dialysis therapy. Typically, a nurse or patient care technician oversees the treatment sessions, assists the patients, and records patient information, such as patient vitals, treatment details, and billing information.

Like other health care facilities, one difficulty that dialysis treatment centers encounter is maintaining patient participation in treatment and, thereby, improving medical outcome. To large extent this is accomplished by providing educational materials to patients. The hope and theory are that well informed patients will pay special attention to their own treatment, not miss appointments, and keep their health care providers apprised of any changes in condition or circumstance not otherwise revealed by routine medical testing. Unfortunately, this entails distributing a large amount of information to patients. However, material regarding each patient's treatment may not always be readily identifiable or accessible when the patient needs or desires it. Furthermore, even where the health care provider is able to provide materials of potential interest, patients still may not feel they are actively involved with the treatment process, and therefore be less motivated to follow suggested treatment.

On another front, the advent of more affordable equipment makes home dialysis an option for many patients, who find it offers them greater privacy, flexibility of scheduling and overall comfort. Home dialysis can also be advantageous to health care providers since it does not require the nursing, equipment and space overhead of standard in-center care. However, even for those patients on a course of home dialysis therapy, maintaining patient participation in treatment can be difficult. An additional difficulty is that of patient monitoring. Home therapy affords fewer opportunities to assess patient health, well-being and treatment compliance. Care givers typically have that opportunity only when patients visit their local dialysis clinic for monthly evaluation—and, then, only to the extent that the success of treatment can be determined from routine testing and from patient reporting.

An object of the invention is to provide improved methods and systems for health care provision.

A related object is to provide such methods and systems as can be used to improve delivery of information in connection with health care provision, both at home and in-center.

A further object is to provide such methods and systems as can be used to increase patient participation and satisfaction with health care provision.

A still further object is to provide such methods and systems as can be used in connection with dialysis treatment and/or otherwise in the provision of health care.

### SUMMARY OF THE INVENTION

The foregoing objects are among those attained by the invention which provides, in some aspects, a method of driving an electronic data interface (e.g., a computer display or a “touch screen”) in connection with medical treatment of a patient. Such a method includes detecting a current medical condition of the patient and driving an electronic data interface in order to (i) deliver content to the patient based on that medical condition, (ii) query the patient based on that medical condition, and/or (iii) stimulate participation of the patient in his or her care based on that medical condition. Interactions with the patient are tailored to improve the clinical outcome of the patient's treatment based on the medical condition through education and/or patient participation.

Related aspects of the invention provide such methods as used in connection with the delivery of hemodialysis and/or peritoneal dialysis treatment of a patient.

Related aspects of the invention provide such methods in which the step of driving the electronic data interface includes providing textual, audio, video and/or other education material to the patient. According to further related aspects of the invention, the step of driving the electronic data interface includes providing questions to and collecting responses from the patient, e.g., as part of a survey, questionnaire, or other interrogatory process. According to further related aspects of the invention, the step of driving the electronic data interface includes hosting a game (e.g., a “video” board or card game) in which the patient participates, e.g., on the touch screen.

According to further aspects of the invention, the step of detecting the current medical condition of a patient includes receiving clinical data regarding the patient's current medical condition from medical apparatus, e.g., a hemodialysis or peritoneal dialysis machine, coupled to the patient. Related aspects of the invention include transmitting clinical data regarding the patient's current medical condition from such medical apparatus to a clinical database system and, further, driving the electronic data interface in accord with information pertaining to such condition received from that system.

Still further aspects of the invention provide methods as described above in which the current medical condition includes a clinical condition of the patient, including, at least one of blood pressure, heart rate, and blood potassium level. According to aspects of the invention practiced in connection with the provision of hemodialysis or peritoneal dialysis, that condition can include other clinical measurements sensed by equipment providing such treatment to the patient.

Further aspects of the invention provide methods as described above in which the step of driving the electronic data interface includes (i) delivering content to the patient based the patient's medical history and/or medical record, (ii) querying the patient based on his/her medical history and/or medical record, and/or (iii) stimulating participation of the patient in his or her care based such medical history and/or medical record.

Still yet further aspects of the invention provide methods as described above that include storing medical records and/or other patient-specific information in a clinical database system.

Yet still further aspects of the invention provide methods as described above in which the step of driving the electronic data interface includes displaying to the patient a plurality of mode and/or content selection options, permitting the patient to select from among those options, and further driving the electronic data interface in accord with the patient selection. According to related aspects of the invention, such a methodology can be used to permit the patient to select among modes for the provision of textual, audio, video and/or other educational material, and, optionally, within that mode, to select specific content to be delivered; the provision of a survey or questionnaire, and, optionally, within that mode, to select a specific survey or questionnaire to answer; and/or the playing of a game or other patient-participatory experience and, optionally, within that mode, to select a specific game to play.

According to further related aspects of the invention, the steps displaying to the patient mode selection options, permitting the patient to select from among those options, and/or further driving the electronic data interface in accord with the patient selection can include restricting and/or expanding such selections based on the patient's medical condition and/or his her compliance with medical treatment requirements, goals, or milestones. Thus, by way of non-limiting example, a patient who has satisfactorily brought down his blood pressure may be entitled to a broader range of mode and/or content options, while a patient who has not satisfactorily brought down his blood pressure may be limited to a restricted range of options.

Further aspects of the invention provide methods as described above in which the step of driving the electronic data interface includes selecting among one or more of the aforementioned modes and/or educational content to be delivered in connection therewith in accord with a preprogrammed algorithm.

Still further related aspects of the invention provide such methods as enable a medical care professional to select among one or more of the aforementioned modes and/or to select educational content to be delivered to the patient in connection therewith.

Yet still further aspects of the invention provide methods as described above including driving the electronic data interface to authenticate the patient before one or more of (i) delivering content thereto based on a medical condition, (ii) querying the patient based on that medical condition, and/or (iii) stimulating participation of the patient in his or her care based on that medical condition.

In another embodiment, a method of delivering patient-specific content includes providing a digital data display device configured to display data to a patient receiving medical treatment and authenticating the patient as a valid user of the digital data display device. The method can further include transmitting first data to the digital data display device from at least one database, wherein the first data is chosen for transmission based on at least real-time data related to medical treatment of the authenticated patient. The authenticated patient can be allowed to choose from the first data additional data for transmission from the at least one database to the digital data display device.

Further aspects of the invention provide medical treatment apparatus and systems operating in accord with the methodologies discussed above. In one such aspect, by way of non-limiting example, the invention provides a patient-specific content delivery system that includes a computer-driven interface device (e.g., a display and/or touch screen) configured to display and/or receive information to/from a patient for purposes of any of (i) delivering content to the patient based on that medical condition, (ii) querying the patient based on that medical condition, and/or (iii) stimulating participation of the patient in his or her care based on that medical condition. Such an interface device can, according to aspects of the invention be configured as part of, or to operate connection with, hemodialysis equipment, peritoneal dialysis equipment, or other patient care equipment.

According to further aspects of the invention, medical treatment apparatus and systems as described above can include a clinical database system in electronic communication that stores information pertaining to real-time medical conditions of a patient undergoing medical treatment. Such a clinical database system can, according to related aspects of the invention, store medical records and other patient-specific information (e.g., medical histories) for use in displaying and/or receiving information to/from the interface device.

The foregoing are among the objects attained by the invention, which provides in some aspects methods of patient care that include detecting a current medical condition of a patient undergoing therapy via a medical treatment apparatus, e.g., an in-center or home dialysis machine. This can be by sensing blood pressure, heart rate, weight, glucose level, hemoglobin level, and/or blood potassium level (all by way of example) using sensors that form part of (and/or that are coupled to the apparatus) and transmitting information regarding that medical condition to a digital data processing system disposed remotely from the medical treatment apparatus. Related aspects of the invention provide such methods that further include delivering to a health care provider—via the remote digital data processing system—current (i.e., up-to-date) medical condition data regarding the patient under treatment. This can include, for example, presenting that data via trend graphs.

Further aspects of the invention provide methods for patient care, for example, as described above, that include acquiring any of a still or video image of the patient and transmitting that image to the remotely-disposed digital data processing system, e.g., for presentation to the health care provider.

In other aspects, the invention provides methods for patient care that include detecting a medical condition of a patient receiving medical treatment by (i) receiving data from a medical treatment apparatus coupled to the patient and/or (ii) accepting from the patient a characterization of any of a medical condition and/or medical treatment. Such a method further includes transmitting information regarding that condition and/or characterization to a digital data processing system disposed remotely from the medical treatment apparatus.

Further aspects of the invention provide methods for patient care, for example, as described above, wherein the data received from the medical treatment apparatus includes data from one or more physiometric sensors, and wherein that data is transmitted to the remotely-disposed digital data processing system. Related aspects of the invention provide such methods wherein the received and transmitted data is from operational sensors coupled to the medical treatment apparatus. This can include, for example, data from sensors reflecting treatment times, medication level/dosing data, and so forth.

Still further aspects of the invention provide methods for patient care, for example, as described above, including generating graphs of one or more data received from the medical treatment apparatus. This can be done, for example, by a processor remote from that apparatus.

Other aspects of the invention provide methods of patient care that include treating a patient with a medical treatment apparatus, detecting one or more operating conditions of that apparatus via sensors coupled thereto, and analyzing data reflecting those operating conditions to any of track inventory and/or use of supplies in connection with treating the patient. The supplies can include any of erythropoietin, dialysis solutions, other medicines, and/or other supplies utilized in connection with such treatment.

Further aspects of the invention provide methods for patient care, for example, as described above, including responding to such analysis by effecting ordering and/or delivery of supplies for use in connection with treating the patient.

In other aspects, the invention provides methods of patient care that include treating a patient with a medical treatment apparatus, detecting operating conditions of that apparatus and/or a current medical condition of the patient, generating an alert based on one or more of those conditions, and displaying that alert, along with a workflow of actions to be taken in response thereto by a health care provider.

Further aspects of the invention provide methods for patient care, for example, as described above, including responding to selection of a displayed action (e.g., by a nurse or other health care provider) by initiating that action. This can include, for example, contacting a patient, contacting a health care provider and making a notation in a patient record. Further aspects of the invention provide methods for patient care, for example, as described above, wherein the alerts and/or display thereof is executed remotely from the medical treatment apparatus.

Still further aspects of the invention provide medical treatment apparatus and systems operating in accord with the methodologies discussed above.

These and other aspects of the invention relating to patient care are evident in the drawings and in the detailed description that follows.

### BRIEF DESCRIPTION OF THE DRAWINGS

A more complete understanding of the invention may be attained by reference to the drawings, in which:

FIG. 1 depicts a plurality of digital data display devices according to the invention, as well as a plurality of medical treatment apparatus (e.g., dialysis machines) with which such devices may be used;

FIG. 2 depicts a schematic diagram of a database system according to the invention that stores data for display on a digital data display device;

FIG. 3 depicts an authentication screen display of a digital data display device according to the invention that facilitates authentication of a patient that may use the digital data display device;

FIG. 4 depicts a menu screen display of a digital data display device according to the invention that facilitates selection of data for display;

FIG. 5 depicts a television screen display of a digital data display device according to the invention that facilitates selection of data for display;

FIG. 6 depicts an education screen display of a digital data display device according to the invention that facilitates selection of data for display;

FIG. 7 depicts a music screen display of a digital data display device according to the invention that facilitates viewing of data;

FIG. 8 depicts a games screen display of a digital data display device according to the invention that facilitates viewing of data;

FIG. 9 depicts an Internet screen display of a digital data display device according to the invention that facilitates viewing of data;

FIG. 10 depicts a survey screen display of a digital data display device according to the invention that facilitates viewing of data;

FIG. 11 is a flow diagram illustrating operation of a system according to the invention;

FIG. 12 depicts a schematic diagram of system according to an invention that facilitates selection and transmission of data for display on a digital data display device;

FIG. 13 depicts a patient treatment station in accord with the teachings hereof; and

FIG. 14 depicts screens generated by a system according to the invention to reflect a workflow following issuance of an alert

### DETAILED DESCRIPTION OF THE ILLUSTRATED EMBODIMENT

Described herein are methods, systems and devices for medical treatment and patient care, particularly, by way of non-limiting example, in the context of dialysis centers (including, as used here, hospitals or other central treatment centers) and in the context of homes—though applicable in a range of health care settings. Those methods, systems and devices, which provide for improved clinical outcome of patient care by facilitating generating alerts to, and communications of information with, a health care provider. Those outcomes are also improved through education and/or patient participation, include driving a digital data interface (e.g., a computer display or touch screen) accessible by the patient in accord with that patient's current medical condition. More specifically, that interface can be driven to (i) deliver content to the patient based on a medical condition of the patient, (ii) query the patient based on that medical condition, and/or (iii) stimulate participation of the patient in his or her care based on that medical condition (collectively, “condition-based information transfer”), all in accord with the patient's blood pressure, potassium levels, weight, glucose level, hemoglobin level or other medical conditions.

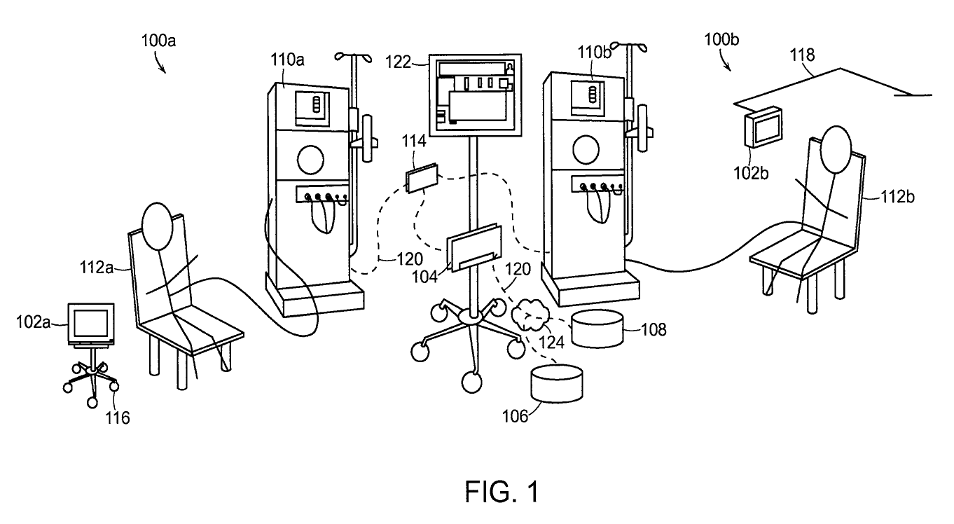

FIG. 1 illustrates patient treatment stations 100a, 100b according to one practice of the invention. Although two such stations are shown in the drawing, other embodiments may utilize a lesser or greater number of stations. For example, embodiments of the invention intended for the home may utilize only one such station.

The patient treatment stations 100a, 100b comprise an electronic data interface (e.g., a display screen, a computer driver interface, or a touch screen) 102a, 102b that provides a visual and possibly tactile interface with a user and a digital processor 104 that controls the display screen 102a, 102b (e.g., vis-à-vis the display of prompts, as well as the input, display, communication, collection and/or storage of the information therefrom and thereto), and that interfaces with other such database systems 106, 108 (data storage mechanisms such as databases, servers, or otherwise), as well as with medical treatment apparatus, such as dialysis machines 110a, 110b (hemodialysis or peritoneal dialysis treatment). The patient treatment station 100a, 100b does not necessarily require a physical keyboard for user input, thereby greatly reducing the risk of patient infection. In addition, the display screen 102a, 102b interface and the method of condition-based information transfer can allow customized content to be automatically and/or manually chosen by a patient 112a, 112b for delivery to the patient 112a, 112b in real-time based on a medical condition of the patient 112a, 112b at the machines 110a, 110b.

The condition-based information transfer includes one or both of receiving medical condition information from the patient treatment station 100a, 100b and delivering content from the databases 106, 108 to the patients 112a, 112b (via the screens 102a, 102b) based on the received medical condition information. The customized information delivered to the display screens 102a, 102b can include textual, audio, video, and/or other education material that includes clinical data, which can facilitate the improvement of clinical outcome through education and patient participation in their own care, and entertainment, which can improve overall patient satisfaction with their treatment, improve patient compliance with prescribed treatment times, and provide a relaxing atmosphere that can positively affect overall clinical outcomes. The customized information received from the patients 112a, 112b can include medical data automatically gathered from the machines 110a, 110b and/or from other devices coupled to the patients 112a, 112b. The patients 112a, 112b can also transfer information to the processor 104 in response to content on the display screens 102a, 102b, such as choices of additional content for delivery or answers to survey questions.

As shown, the stations 100a, 100b are each associated with a medical treatment apparatus 110a, 110b (in this embodiment, hemodialysis machines) of the type commonly known in the art. The display screens 102a, 102b of the stations 100a, 100b are in electronic communication with the processor 104 (or otherwise coupled thereto) for use by a user such as a patient 112a, 112b being treated with the dialysis machines 110a, 110b, a nurse, a patient care technician, or other health care provider. Although two stations 100a, 100b are shown, those skilled in the art will appreciate that the processor 104 and the storage mechanisms 106, 108 may support more than or less than two stations. Furthermore, the stations 100a, 100b can be in locations remote from each other and/or the processor 104. For example, the stations 100a, 100b can be located in a clinical setting (e.g., a hospital or a hemodialysis clinic) or a nonclinical setting (e.g., at a home of a dialysis patient receiving peritoneal dialysis using a home-based system).

The stations 100a, 100b can each include a touch screen display 102a, 102b, the digital data processor 104, and an adapter 114. The touch screen displays 102a, 102b can each include a conventional device of that type known in the art for visual and/or tactile interface with an operator—here, patient 112a, 112b—operated in accord with the teachings hereof. The units 102a, 102b can be based on liquid crystal display technology, cathode ray display technology, or otherwise. Though the illustrated embodiment relies on color display technology, other embodiments may utilize monochrome (e.g., employing shading, hashing, or other visual indicators in place of the colorations discussed below). The displays 102a, 102b are sized and provides resolution sufficient to display and collect information of the type described or are otherwise suitable for the digital data environment for which it is used. Preferably the displays 102a, 102b can be adapted for ready cleaning and/or sanitization, particularly when used in a clinical environment where multiple people typically use the displays 102a, 102b.

Additionally, while the displays 102a, 102b preferably include touch screens, the display devices 102a, 102b can include any device capable of displaying information to a user, e.g., a personal computer, a television, a portable digital device, or any other electronic display device. Furthermore, the displays 102a, 102b can have any configuration where they may be made easily, comfortably accessible to the patient 112a, 112b, such as on a rolling stand 116 (left display 102a), on an adjustable arm 118 (right display 102b), or otherwise. In other embodiments, the displays 102a, 102b may be more fully portable (e.g., lightweight and with carrying handles), fixed (e.g., wall-mounted or cabinet-mounted) or otherwise—all in the conventional manner of clinically-deployed medical data entry devices.

The displays 102a, 102b can be adapted to provide an ergonomic work station such that data entry puts a minimal stress on the patients 112a, 112b. The height and angle of the displays 102a, 102b can facilitate data entry and minimize the risk of repetitive stress disorders. The height and angle of the displays 102a, 102b also can make their screens less visible to a casual onlooker such as another patient or ambulance driver walking by, which helps protect the confidentiality of any patient data being displayed. The illustrated displays 102a, 102b can be set to display a neutral screen saver after a set or variable amount of time of system inactivity to further protect the confidentiality of such patient data.

The digital data processor 104 can include an embedded processor, personal computer, workstation, minicomputer, mainframe or other digital data processing device of the type known in the art, as adapted in accord with the teachings hereof. The digital data processor 104 may be a stand alone device or may be integral to one or more other components of the illustrated system, e.g., the touch screens 102a, 102b and/or medical treatment apparatus 110a, 110b. It may, moreover, be coupled for communication over communication links 120 with the touch screen displays 102a, 102b and the adapter 114 via any combination of wireless connection (e.g., BlueTooth, 802.1x, or otherwise), wired connection (Ethernet, backplane bus, or otherwise), or otherwise, all in the conventional manner known in the art.

Communication on one or more of the communication links 120 (which may include more or fewer linked connections than those shown in FIG. 1) may be secured with a mechanism such as IP security (IPsec), Transport Layer Security/Secure Socket Layer (TLS/SSL), wireless TLS (WTLS), secure Hypertext Transfer Protocol (SHTTP), or any other security mechanism as would be appreciated by those skilled in the art.

The processor 104 can also be in communication with a data entry device such as a touch screen 122 that provides a visual and tactile interface with an administrator, e.g., a nurse, patient care technician, or other medical personnel. Through the touch screen 122, a user can coordinate input, display, communication, collection, and/or storage of data between the displays 102a, 102b, the processor 104, and/or the storage mechanisms 106, 108. Although only one touch screen 122 is shown in this embodiment, there may be any number of such data entry devices. In the illustrated embodiment, the touch screen 122 operates, e.g., for purposes of data input and display, in the manner described in incorporated-by-reference U.S. patent application Ser. Nos. 60/732,335 and 11/591,438, published as PCT/US 2006/042650 and incorporated by reference herein, though, it may operate otherwise in other embodiments.

The database systems 106, 108 can each include a database, a data queue, a buffer, a local or remote memory device, random access memory (RAM), a cache, a server, or digital data storage device of the type known in the art, as adapted in accord with the teachings hereof. The databases 106, 108 are adapted to communicate with the displays 102a, 102b (via the processor 104) over one or more communication links 120 and possibly over a network 124, as described herein. Although the storage mechanisms 106, 108 are shown as separate elements from the processor 104 in this illustrated embodiment, the storage mechanisms 106, 108 can be integral to the processor 104, or the storage mechanisms 106, 108 can otherwise be combined into one storage mechanism or separated into one or more other storage mechanisms. Furthermore, the databases 106, 108 may communicate using the same or different network 124, which can itself include one or more interconnected networks. One or both of the storage mechanisms 106, 108 may be secured using a variety of techniques, as those skilled in the art will appreciate.

In the illustrated embodiment, operation of the stations 100a, 100b in general, and of the touch screens 102a, 102b in particular, are controlled by the processor 104. To this end, and to the extent that this description attributes control and data processor functionality to the touch screens 102a, 102b, it will be appreciated that such control and data processing is provided indeed by the processor 104. Similarly, control and data processing of the storage mechanisms 106, 108 is provided indeed by the processor 104.

The adapter 114 provides communication coupling between the digital data processor 104 (and the storage mechanisms 106, 108) and the medical treatment apparatus (here, dialysis machines) 110a, 110b. In the illustrated embodiment, the adapter 114 is a Universal Serial Bus (USB) hub of the conventional type known in the art. In other embodiments, the adapter 114 can take on other form factors (electrical and/or physical), such as Ethernet, serial cabling, and so forth, suitable for transmitting data to/from the processor 104 and the apparatus 110a, 110b and/or the display 102a, 102b. Moreover, the illustrated adapter 114 can be supplanted by, or supplemented with, wireless communications (e.g., based on BlueTooth, 802.1x, and so forth), consistent with the aforesaid purpose. Regardless, the adapter 114 transmits data in a common protocol defined between the processor 104 and the treatment apparatus 110a, 110b. In the illustrated embodiment, the adapter 114 is a standalone device that is coupled with the processor 104 and the apparatus 110a, 110b via cabling, as shown, though in other embodiments it may be integral with one of more of the other system components (e.g., the processor 104, the storage mechanisms 106, 108, and/or the apparatus 110a, 110b).

In the illustrated embodiment, the displays 102a, 102b are used in connection with the medical treatment apparatus 110a, 110b to facilitate medical treatment (here, dialysis treatment) of the patients 112a, 112b, as shown. Though the illustrated patients 112a, 112b are shown in chairs, those skilled in the art will appreciate that patients can receive treatment in prone or other positions, as well—all in the conventional manner known in the art.

It will be appreciated that FIG. 1 depicts embodiments of systems and devices according to some practices of the invention, and that other embodiments may have lesser, more and/or other elements than those shown in the drawing and described herein. Thus, for example, embodiments of the invention may have greater or fewer patient treatment stations and/or may be capable of treating greater or fewer patients. By way of further example, embodiments of the invention may utilize greater or fewer displays 102a, 102b, touch screens 122, processors and so forth. By way of further example, the functionality of various ones of the elements illustrated and described herein may be incorporated into and/or otherwise combined with other elements.

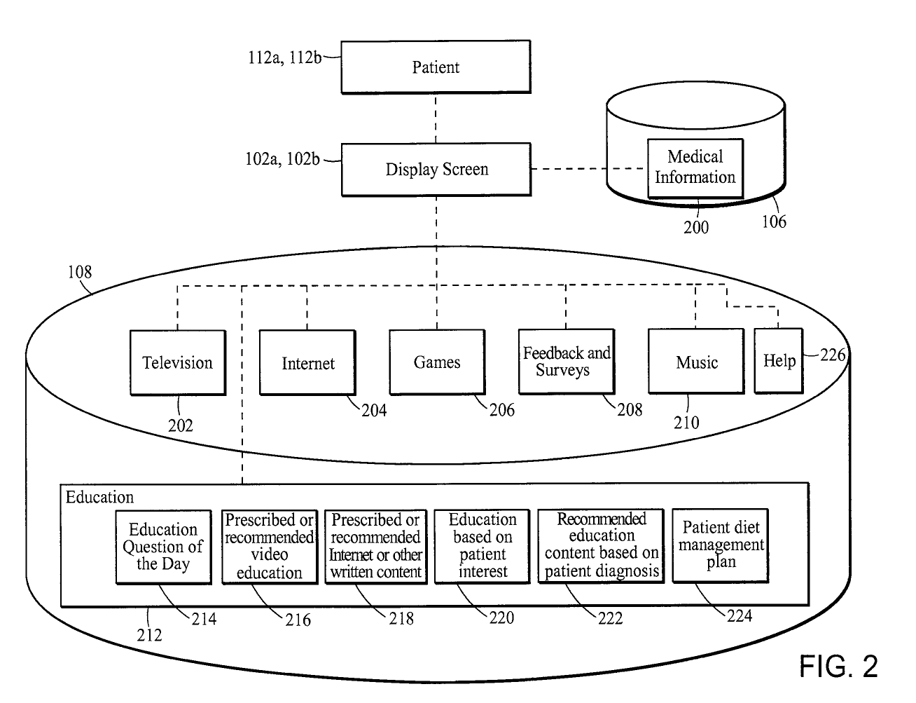

Referring to FIG. 2, the storage mechanisms 106, 108 are shown in more detail. Information stored in one or both of the storage mechanisms 106, 108 can be delivered to the patients 112a, 112b via the display screens 102a, 102b (and the processor 104). The delivered information can be customized for each patient 112a, 112b, chosen in real-time (e.g., on the fly) as the patient 112a, 112b is receiving, will soon receive, or has just received, medical treatment at the machines 110a, 110b. The delivered data can also be prescribed for the patients 112a, 112b by medical personnel before treatment and delivered after patient logon to the system.

As shown, each of the storage mechanisms 106, 108 can include information of different types. The information can be input into the storage mechanisms 106, 108 from a variety of sources. By way of non-limiting example, data can be manually input into the storage mechanisms 106, 108 by a user (e.g., at the touch screen 122), obtained from another database including the other one of the databases 106, 108, received from a monitoring device such as the machine 110a, and other similar data entry techniques.

One database 106 (“clinical database system 106” or “medical database 106”) can store medical records and other patient-specific information 200 while the other database 108 (or “content database 108”) can store content information for delivery to the display devices 102a, 102b. The medical information 200 in the medical database 106 can include programmed data and/or current treatment data (or “treatment information”), e.g., the patient's medical condition-based information (e.g., from the physiometric sensor readings), video and/or still images of the patient (e.g., from the web camera), survey and other query responses, operational sensor readings and/or alerts, etc., from the respective patient treatment stations, as well as from the touch screen 122. Programmed data generally includes data related to patient identification and previous patient care (e.g., patient medical record data). Non-limiting examples of programmed data (generally, etiology data) include patient name, patient medical history, medical treatment history, lab results, treatment plans, dietician patient plans, ethnicity, demographics, and other types of similar data.

Current treatment data generally includes data gathered in real-time while the patient 112a, 112b is being treated at the machines 110a, 110b, e.g., vital signs, dialysis results and performance, information on kidney function, phosphorous levels (all of which may be gathered, e.g., by sensors 1328A-1328D and/or sensors 1330A-1330D), query responses and/or other survey results, video (e.g., surveillance using a video or still-image web camera coupled to the touch screen 102a that can provide retinal tracking to monitor patient activity), etc. More generally, as discussed above, current treatment data can include patient's medical condition-based information (e.g., from the physiometric sensor readings), video and/or still images of the patient (e.g., from the web camera), survey and other query responses, operational sensor readings and/or alerts, etc., from the respective patient treatment stations, as well as from the touch screen 122. Current treatment data can be stored in the medical database 106 to create an archive of medication treatment information. The current treatment data (archived and/or from the instant treatment session) can be used by the processor 104 to determine what data from the content database 108 to deliver to a particular patient.

The content database 108 generally includes information that can be displayed on the display screens 102a, 102b, as further discussed below. The information can be dynamically chosen through analysis by the processor 104 such that the patients 112a, 112b can view information most relevant to their current medical status. Non-limiting examples of information that can be stored for delivery in the content database 108 include television data 202, network data 204 (in which case the content database 108 acts similar to a content server), games data 206, feedback and survey data 208, music data 210, education data 212, and other similar types of data. The education data 212 generally includes information that may be useful in educating the patients 112a, 112b about their medical condition and/or medical treatment. For example, the education data 212 can include an education question of the day 214, prescribed or recommended video education 216, prescribed or recommended Internet or other written content 218, education based on patient interest 220, recommended education content based on patient diagnosis 222, patient diet management plan data 224, and other types of similar data. The content database 108 can also include help information 226 related to use of the touch screens 102a, 102b, the machines 110a, 110b, and/or to data available to users through the touch screens 102a, 102b. Data can be categorized any way in the content database 108, including no categorization at all and categorization as more than one data type (e.g., as education data 212 and as games data 206).

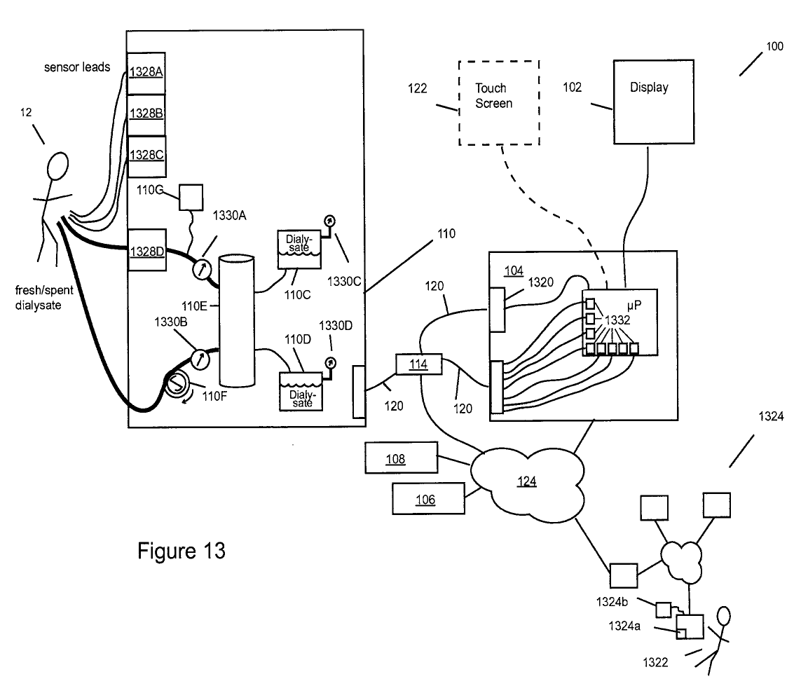

FIG. 13 illustrates a patient treatment station 100 (e.g., representing stations 100a and/or 100b shown in FIG. 1) according to one practice of the invention. Other patient treatment stations may be similarly constructed or otherwise. As above, the illustrated station comprises inter alia a medical treatment apparatus 110 (e.g., representing apparatus 110a and/or 110b shown in FIG. 1), digital data processor 104, touch screen 122, all coupled as shown. In the illustrated embodiment, digital data processor 104 is disposed locally with respect to digital data processor 104 (and the patient), as shown.

As above, it will be appreciated that FIG. 13 depicts embodiments of systems and devices according to some practices of the invention, and that other embodiments may have lesser, more and/or other elements than those shown in the drawing and described herein. Thus, by way of non-limiting example, embodiments of the invention may not utilize touch screens 122 and/or may incorporate the functionality ascribed thereto herein to other elements, such as input/output devices 1324B coupled to processor 1324.

Illustrated medical treatment apparatus 110 comprises a dialysis unit and, more particularly, a hemodialysis unit of the type commercially available or otherwise known in the art for such purpose, e.g., including, for example, fresh and spent dialysate containers 110C, 110D, filter/dialyzer 110E, pump 110F, supplemental medication supply 110G (e.g., for heparin and/or other medications), and so forth, as adapted in accord with the teachings hereof. The illustrated medical treatment apparatus 110 is, moreover, of the type adapted for use in the home or other locale remote for a dialysis center, hospital or other central treatment center. Alternatively, or in addition, the apparatus 110 may be adapted for use in such a dialysis center, hospital or other treatment center. This may, for example, at a location in such a center remote from a physician, nurse, patient care technician or other health care provider—or it may be nearby such a provider. In other embodiments apparatus 110 may comprise a peritoneal dialysis machine or other medical treatment apparatus of the type known in the art or otherwise, again, as adapted in accord with the teachings hereof.

Digital data processor 104 comprises a microprocessor (“μP”) or other processing unit of type commercially available or otherwise known in the art, as adapted in accord with the teachings hereof The digital data processor 104 may be integral to medical treatment apparatus 110 or, as shown here, it may separate therefrom and coupled for communication therewith, e.g., via links 120 and adapter 114, or otherwise. As above, digital data processor 104 is coupled display 102 (e.g., representing displays 102a and/or 102b shown in FIG. 1), as with touch screen 122. Digital data processor 104 may also coupled with keyboard or other input device, e.g., mouse, touch-screen, touchpad, etc. (not shown), or output device, all of the type commercially available in the marketplace or otherwise known in the art, again, as adapted in accord with the teachings hereof. The touch screen 122, display 102 and/or other input and output devices may be integral to medical treatment apparatus 110 or separate therefrom (as shown here).

Digital data processor 104 is coupled via network interface 1320 to support communications with a health care provider 1322, e.g., a physician, nurse, patient care technician, administrator, etc. via digital data processing system 1324 (sometimes referred to herein, simply, as “processor 1324”) and links 120 and network media 124, e.g., as shown. That media 124 may comprise IP, telephone and/or other networks of the type known in the art—wired, wireless and/or otherwise. Network interface 1320 comprises a modem, network interface card and/or other functionality of the type commercially available in the marketplace or otherwise known in the art suitable for supporting communications over network 124, as adapted in accord with the teachings hereof. Examples include cable modems, cellular telephone modems, USB modems, Ethernet cards, and combinations thereof, just to name a few. It will be appreciated that some of that functionality for supporting communications with care provider 1322 via digital data processor 104 and network media 124 may reside on processor 104, as in the case, for example, of operating system drivers, network protocol stacks, and so forth. Together, this functionality (i.e., hardware and/or software) is referred to, here, as “communications logic.”

Processor 104 is also coupled with sensors 1328 (by way of non-limiting example, via communications links 120 and adapter 114) that take biometric readings of the patient 112 to determine his or her medical condition, e.g., in real-time. These “physiometric sensors,” as they are referred to herein, can include blood pressure sensors, heart rate sensors, and so forth. In the illustrated embodiment they are of the type commercially available in the marketplace or otherwise known in the art, as adapted in accord with the teachings hereof. Those sensors may be integral to apparatus 110, processor 104, disposed at station 100, or otherwise. In the illustrated embodiment, the sensors include a heart rate (or pulse) sensor 1328A, a blood pressure sensor 1328B, a weight sensor 1328C and a electrochemical sensor 1328D (e.g., for sensing blood urea levels, ammonia levels, blood potassium levels, hemoglobin levels, and so forth). Sensors 1328A-1328C are shown coupled to the patient, while sensor 1328D is coupled in the blood fluid-flow path of apparatus 110. Other embodiments of the invention may include a greater or fewer number of such sensors, coupled to the patient, the flow-path of apparatus 110, etc., as shown or in other ways known in the art, as adapted in accord with the teachings hereof.

Processor 104 is also coupled to operational sensors 1330 that sense operating conditions of the station 100, apparatus 110, and/or components thereof. These can include sensors that measure the rates of fluid flow, fluid temperature, level, and composition, power conditions, maintenance status and so forth, all by way of non-limiting example. In the illustrated embodiment they are of the type commercially available in the marketplace or otherwise known in the art, as adapted in accord with the teachings hereof. Those sensors may be integral to apparatus 110, processor 104, disposed at station 100, or otherwise. In the illustrated embodiment, the sensors include an inflow-rate or pressure sensor 1330A, and outflow-rate or pressure sensor 1330B, fresh dialysate level sensor 1330C, spent dialysate level sensor 1330D. Additional sensors (not shown) can, by way of non-limiting example, sense treatment times (e.g., on/off times of the apparatus 110), levels or dosings of fresh and spent dialysate containers 110C, supplemental medication supply 110G, and/or of other consumables utilized in connection with treatment. A further such sensor may comprise the aforementioned web camera. In other embodiments, other operational sensors of the type suggested above are provided instead or in addition to those shown in the drawing and/or discussed here.

Processor 104 can include monitoring logic, here, represented by modules 1332, that monitor the biometric readings of the patient, operational states of station 100 and/or apparatus 110, as well as detect alert or alarm (terms which are used interchangeably herein) conditions, e.g., as reported directly from components of station 100 and/or apparatus 110 and/or when readings from the physiometric sensors 1328 and/or operational sensors 1330 fall outside of selected ranges. That monitoring logic can also, by way of non-limiting example, monitor when and for how long the various sensor readings, for example, are outside those ranges.

Thus, for example, the monitoring logic 1332 can detect the incidence and duration of systolic blood pressure readings below physician—(or other care provider—) defined levels, as well as changes (deltas) in blood pressure greater than such levels. By way of further example, the monitoring logic can detect when medications are administered and in what quantity. By way of still further example, the monitoring logic can detect when treatment sessions are initiated and how long they are run; the incidence and duration of “door ajar” alerts, “filter replacement” alerts, “dialysate replacement” alerts, and/or other alerts or conditions reflecting whether proper and efficient care is being delivered to the patient 112, and so forth. Though illustrated here has modules (i.e., software and/or hardware) executing on/forming part of processor 104, in other embodiments, monitoring logic 1332 can be form part of apparatus 110 and/or may be disposed at station 100, or otherwise.

Illustrated digital data processor 104 is coupled to digital data processing system 1324, e.g., via interface 1320 and network link 124. That system 1324 may comprises one or more cell phones, smart phones, personal digital assistants, computers, or other devices suitable for communicating with digital data processor 104 to receive the patient's treatment information, including, for example, medical condition-based information (e.g., real-time physiometric sensor readings), operational sensor readings and/or alerts of the type described above. In the illustrated embodiment, the system 1324 is disposed remotely from station 100 (and from digital data processor 104).

Moreover, in the illustrated embodiment, the system 1324 comprises a computer system of the type commonly available in a hospital, dialysis center or other central location of the type that employs or otherwise physicians, nurses, patient care technicians or other health care providers to oversee treatment of patients, all as adapted in accord with the teachings hereof. In other embodiments, the system 1324 is located with the station 100 at the same facility (e.g., in a hospital, dialysis center or other central location) and/or in the same department, floor, ward and/or room of such a facility, by way of non-limiting example, and coupled with computer 104 by way of a bus, network or other media, or combination thereof, wired or wireless, dedicated, shared or otherwise.

The illustrated system 1324 also includes an analysis/reporting module 1324A, implemented in software on one or more of the computers or other computational devices making up that system, that analyzes and/or reports the information collected and transmitted by processor 104 in regard to station 100. That data can include treatment data, e.g., data gathered, e.g., by sensors 1328A-1328D and/or sensors 1330A-1330D, alerts generated by monitor modules 1332, by processor 104, or otherwise, query/survey results, video (or still images) from the web camera coupled to the touch screen 102a, 102b. The system 1324 also includes a touch screen 1324B (or other combination of input and/or output devices) of the type commercially available in the marketplace or otherwise known in the art (as adapted in accord with the teachings hereof) for presenting results generated by module 1324A. However, in the illustrated embodiment, the touch screen 1324B operates, e.g., for purposes of data input and display, in the manner described in incorporated-by-reference U.S. patent application Ser. Nos. 60/732,335 and 11/591,438, published as PCT/US 2006/042650 and incorporated by reference herein, though, it may operate differently in other embodiments.

Illustrated system 1324 may be coupled to database systems 106, 108, e.g., via network 124. The system 1324 can execute database management software (not shown) that stores the patient's medical condition-based information (e.g., real-time physiometric sensor readings), operational sensor readings and/or alerts, etc., to the database systems 106, 108 and/or that retrieves that information therefrom, e.g., for reporting and other purposes consistent with the teachings hereof. In these regards, the database management software can be of the type commercially available or otherwise known in the art for such purpose, as adapted in accord with the teachings hereof.

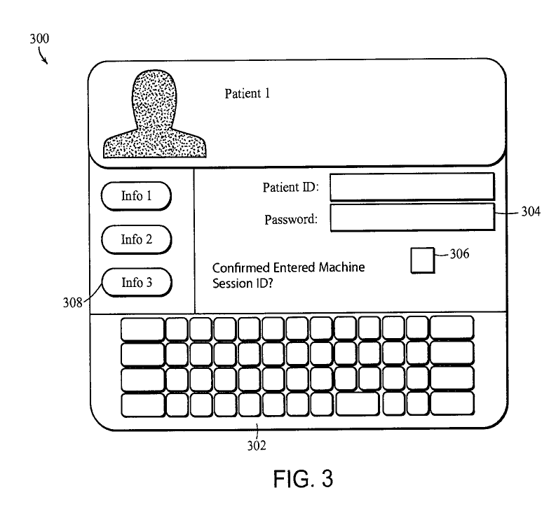

Referring to FIG. 3, in the illustrated embodiment, the touch screens 102a, 102b operate under control of the processor 104 to provide an entry screen 300 on the touch screens 102a, 102b, which the patients 112a, 112b (and/or medical personnel such as nurses and patient care technicians) can use to input letters, numbers, and/or other symbols via a keyboard icon 302 to be authenticated as a valid user and thereby access content from the processor 104 and/or the databases 106, 108 through the touch screens 102a, 102b. The entry screen 300 may or may not occupy a substantial entirety of the display screen 102a, 102b (e.g., to the exclusion of other screens), as with all screens described herein.

Although authentication is illustrated in this example as occurring by the patients 112a, 112b at the touch screen 102a, 102b, the patients 112a, 112b may be authenticated in a similar way elsewhere (e.g., through another device in electronic communication with the touch screens 102a, 102b and/or the processor 104) and by a user other than the patients 112a, 112b (e.g., an administrator at the display screens 102a, 102b or the touch screen 122). Additionally, those skilled in the art will appreciate that authentication may occur in a multitude of other ways, e.g., using identification techniques such as voice imprint, fingerprint, retinal scan, biometric identification, though an access card, etc.

As shown in FIG. 3, the touch screens 102a, 102b can additionally, or alternatively, display text boxes 304, check boxes 306, button icons 308, or other widgets that a user can use to make input designations on the entry screen 300. Together, the icons 302, 304, 306, 308 (and/or any others displayed on the screen 102a, 102b) can be used to select and/or enter patient (user) identification. Furthermore, the icons 302, 304, 306, 308 (and/or any others displayed on the screen 102a, 102b) can be organized in any configuration on the screens 102a, 102b, including different configurations on different ones of the screens 102a, 102b in communication with the processor 104. After authentication as a valid user, a user may be able to enter additional information through the entry screen 300, such as treatment information (e.g., blood pressure, pulse, treatment parameters) and post-treatment information (e.g., next scheduled patient visit, drug prescriptions, and so forth).

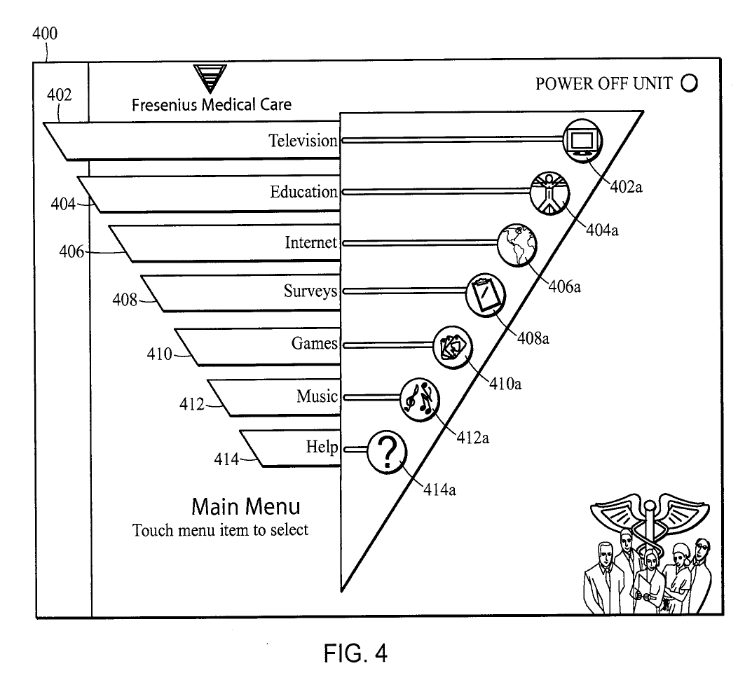

FIG. 4 illustrates a menu screen 400 driven by the processor 104 to the touch screens 102a, 102b, e.g., at the start of a treatment session following user authentication (or before, as user authentication may occur after an initial selection on the menu screen 400, to facilitate selection of content for display on the touch screens 102a, 102b). The menu screen 400 displays a plurality of mode selection options 402, 404, 406, 408, 410, 412, 414 (e.g., content corresponding to data types in the medical database 106 and/or the content database 108) with a corresponding graphic 402a, 404a, 406a, 408a, 410a, 412a, 414a disposed adjacent to its corresponding text option 402, 404, 406, 408, 410, 412, 414 in the list. By way of non-limiting example, in the illustrated embodiment, the operator selects a content option by touching the options graphic 402a, 404a, 406a, 408a, 410a, 412a, 414a. However, those skilled in the art will appreciate that the method for selecting content from the list may vary (e.g., the user may touch the options text bar 402, 404, 406, 408, 410, 412, 414, use a peripheral device such as keyboard or mouse, or speak the options text if the display screen 102a, 102b has voice recognition capability).

FIGS. 510 illustrate embodiments of various screens that can display based on the patient's content choice on the menu screen 500 (or on another screen since mode and content choices may be made available on various screens). The screens illustrated herein are provided as non-limiting examples; screens can have any configuration and one or more additional screens may be provided to the patients 112a, 112b before or concurrent with these content-specific screens, e.g., required viewing content as further described below. As noted above, operation of the devices 102a, 102b on which these screens are displayed (and on which the patient or other user provides input) are controlled by the processor 104. To this end, and to the extent that this description attributes control and data processor functionality to the touch screens 102a, 102b, it will be appreciated that such control and data processing is provided indeed by the processor 104. Similarly, control and data processing of the storage mechanisms 106, 108 is provided indeed by the processor 104.

The screens 400, 500, 600, 700, 800, 900, 1000 can each include one or more navigational features (in addition to or instead of those described for specific ones of the screens 400, 500, 600, 700, 800, 900, 1000) such as a home button 506 to access the menu screen 400, a back button 508 to return to the previously viewed screen, a help button 510 (which may provide help regarding the currently viewed screen and/or other features of the touch screen 102a, 102b and its user interface), and other similar types of navigational features. The navigational features can be located anywhere on the screens 400, 500, 600, 700, 800, 900, 1000. Onscreen instructions 516 particular to the currently viewed screen may or may not be provided by default. Scroll buttons (e.g., page up/down buttons, previous/next buttons) can be provided and used if more options are available for selection than can be contemporaneously displayed on any given screen. Any selected option on any screen can open a screen in a new window or in the same window as the screen from which it was selected. Based on the option selected, a user input mechanism may appear on (or disappear from) the screen to ease use of the selected application (e.g., a keyboard appears for use with a crossword puzzle or a numeric keypad appears for use with a multiple choice-based survey).

Color schemes and/or images on the display screen 102a, 102b can be used to help reduce the chance of operator error. For example, in the illustrated embodiment of the menu screen 500, the graphics 402a, 404a, 406a, 408a, 410a, 412a, 414a have different images, e.g., a television 402a for television content and cards 410a for games content. In the illustrated embodiment of the menu screen 500, the text options 402, 404, 406, 408, 410, 412, 414 have the same color scheme, but each content choice can have a different color scheme. The color scheme can be used as a reference by the patient 112a, 112b during use of the touch screen 102a, 102b for determining/confirming which content he/she has selected, e.g., by having a screen background color match the chosen option's color scheme.

One skilled in the art will appreciate that a variety of patient information can be entered with the methods, systems and devices described herein and that the display on the touch screen 102a, 102b can be adapted depending on the intended use. To facilitate data entry and reduce risk of operator error using the touch screen 102a, 102b, the processor 104 can render only necessary keypad and/or button icons on the touch screen 102a, 102b. Thus, where data entry for an input field selected by the operator (or otherwise activated) requires only numeric values, the processor 104 can renders only a numeric keypad on the display 102a, 102b. Conversely, where an alphanumeric input field is selected by the operator (or otherwise activated), the processor 104 can renders a full (alphanumeric) keypad on the display 102a, 102b. As the operator moves from one data entry type to another, the processor 104 can changes the icons (or data entry widgets), as necessary, even on the same screen. For example, when a text entry widget is activated, a keyboard can be rendered by the processor 104 on the touch screen 102a, 102b. Conversely, when a numeric entry field is activated, a keypad can be rendered.

The screens discussed herein are not limited to any particular layout or configuration. For example, manipulation tools such as touch-activated icons, pulldown menus, tabs, buttons, selection boxes, and scrollbars can be implemented using any type of manipulation tool. Furthermore, two or more screens may be combined and presented on a single screen and one screen may be divided into two or more screens. There may also be additional screens. Furthermore, users may manipulate the screens in any way, e.g., using a mouse, a touch screen, a stylus, keyboard commands, etc. For example, a user may move his or her mouse pointer over an icon and click on the icon to access a particular functionality.

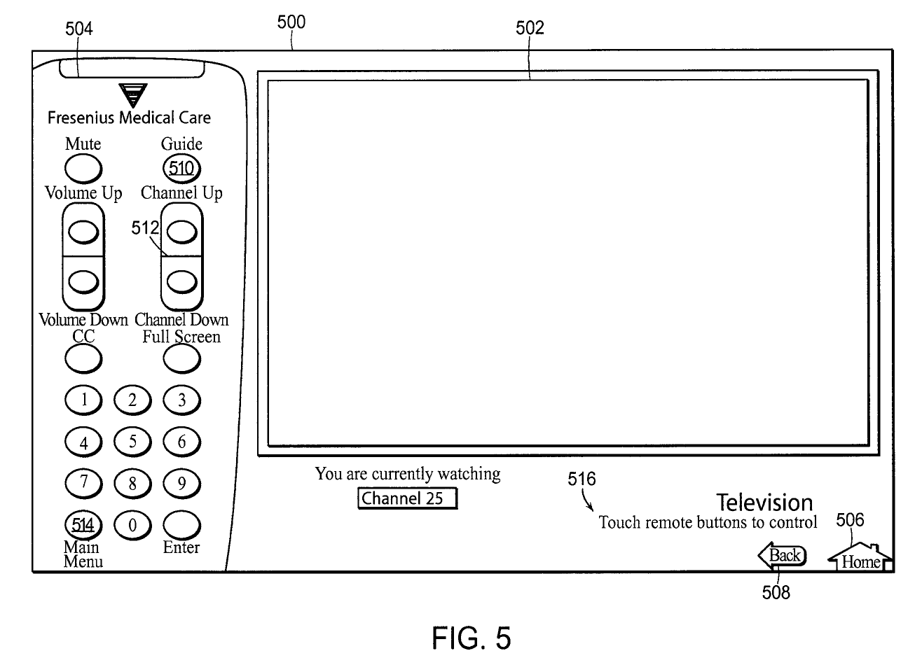

Referring first to FIG. 5, upon selection of the television text 402 or graphic 402a, the touch screen 102a, 102b can display a television viewing screen 500. The television viewing screen 500 can display a television screen 502 that shows television channels like an ordinary television, pre-stored television programs, and the like. Content for the television screen can be delivered to the touch screen 102a, 102b from the television data 202 (FIG. 2) or from another source, e.g., a cable or satellite connection.

The television viewing screen 500 can be programmed to initially display to the patient 112a, 112b on a specified channel or with a specified video/audio introduction, or the patient 112a, 112b may be able to immediately choose television content on the television viewing screen 500. The patient 112a, 112b can choose content for display on the television screen 502 by, for example, touching buttons on a remote control style menu 504 on the television viewing screen 500. The remote control 504 can include, among other options, channel up/down buttons 512 and a main menu button 514 to access a television home page (e.g., a guide of available channels). Content 202 for display on the television screen 502 is typically the same for different patients 112a, 112b, but the television data 202 may be based at least on identification of the patients 112a, 112b as described further below, e.g., restricting users under a certain age to particular television channels.

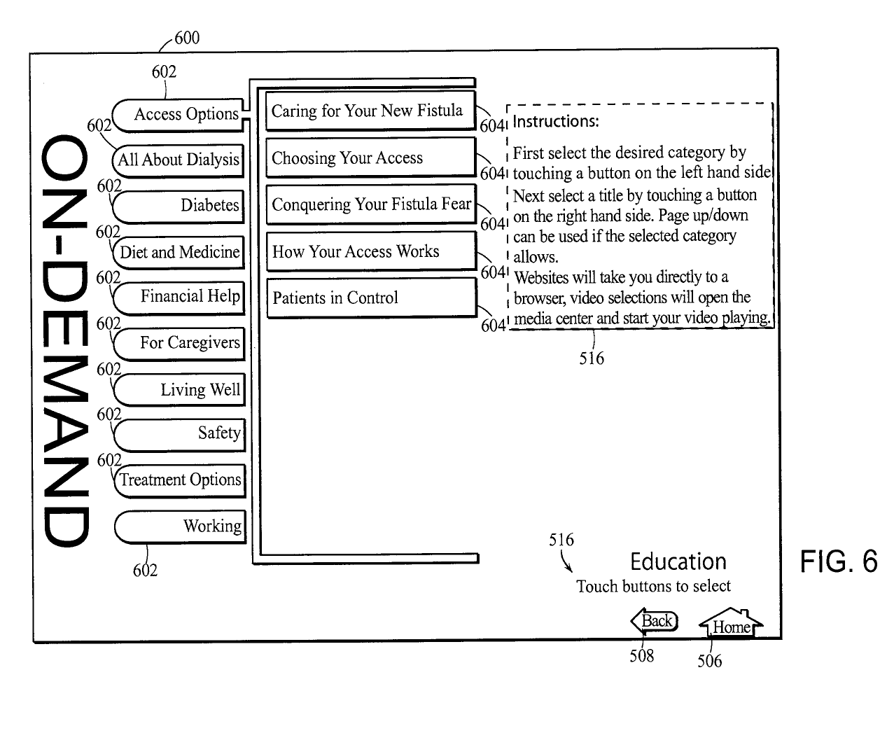

If the education option 404, 404a is chosen (FIG. 4), then the touch screen 102a, 102b can display an education screen 600 as shown in FIG. 6. The education screen 600 can include the education data 212 (FIG. 2) displayed as one or more education content options 602, which can be presented textually or graphically as described above regarding the main menu screen 400. In this example, the education options 602 are shown as a vertical list of text buttons. The education options 602 can be catered to a particular user of the touch screens 102a, 102b (as identified by login via the entry screen 300 of FIG. 3) as described further below.

Non-limiting examples of the education options 602 include treatment information (e.g., dialysis access options, educational information regarding treatment at the dialysis machines 110a, 110b, treatment options, etc.), diagnosed patient conditions (e.g., diabetes, high blood pressure, etc.), self medical care issues (e.g., diet and medicine, living well, safety, etc.), financial help, caregivers, working/employment issues, and similar types of educational information. Upon selection of one of the education options 602, another menu of category specific education options 604 can appear, e.g., on a new screen, on the education screen 600 in tree format to the right of the selected option (as shown), or otherwise. The category specific education options 604 can also be catered to a particular user as explained below. The education option 602 to which the displayed category specific options 604 apply may be highlighted, such as by changing its color, changing its appearance to look as if it has been depressed, or otherwise highlighting the education option 602 (as shown, the selected access options button 602 is ringed in green).

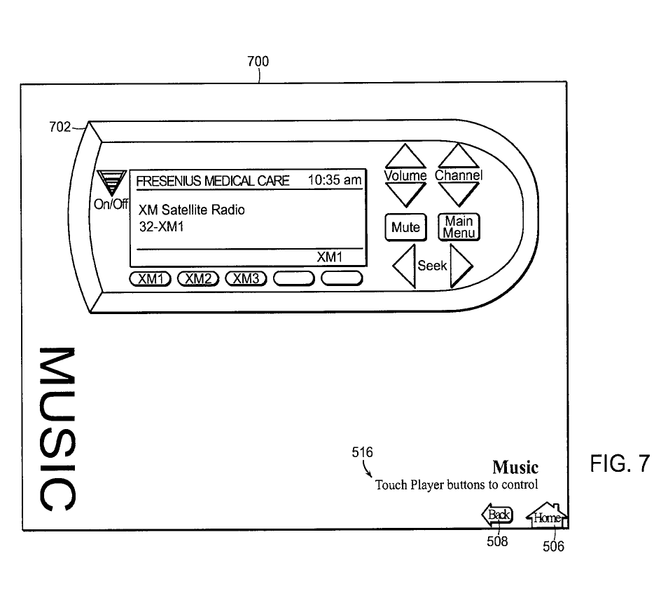

Although not always specifically mentioned with regard to particular screens, category specific options can be available on any screen, e.g., choices of music genres, radio stations, preprogrammed playlists, etc. upon selection of the music option 412, 412a prior to accessing the music screen 700 of FIG. 7.

The music screen 700 can include an audio player 702 which can play music and/or other types of audio. The audio player 702 can play music data 210 from the content database 108 and/or other audio content, such as radio stations (accessed like a traditional radio or over a network such as the Internet) and recorded audio (e.g., compact discs, digital music, etc.). The patients 112a, 112b may be able to provide music content for play through the audio player 702, e.g., by interfacing a portable music player to the display device 102a, 102b.

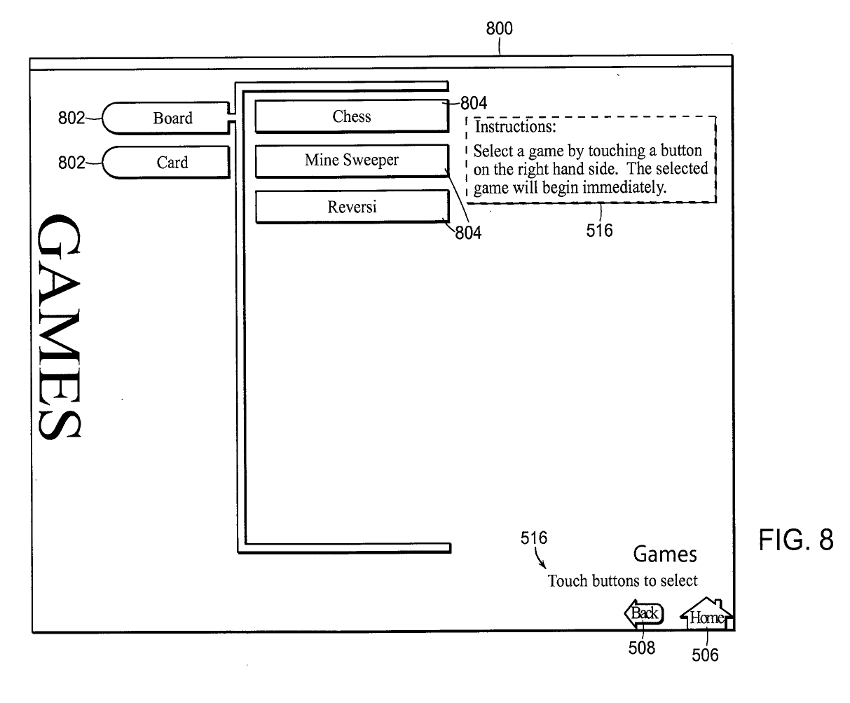

Referring to FIG. 8, if the games option 410, 410a is chosen (FIG. 4), then the touch screen 102a, 102b can display a games screen 800. The games screen 800 in this example initially presents game category options 802 (e.g., board, cards, sports, word, educational, etc.), which when selected trigger display of game options 804 within the selected category. The games screen 800 may not list game category options but instead present a list of all games, organized alphabetically, by approximate game completion time, by favorites as indicated by previous choices by the patient 112a, 112b, or other organizational scheme. The game options 804 can include traditional games (e.g., solitaire, chess, Mine Sweeper, Reversi, hearts, poker, checkers, crossword puzzles, baseball, pinball, etc.) and/or educational games designed to educate in game format (e.g., word searches or Hangman using medical terms, trivia, constructing nutritionally appropriate meals within a certain time limit or given various food options, etc.).

Patients 112a, 112b under a certain age may only receive educational game options or may receive educational game options when they select the education option 404, 404a. When the patient 112a, 112b selects one of the game options 804, the game can start immediately, or rules may first be made available. Additionally, the patients 112a, 112b may be able to play games against each other, e.g., coordinated and/or hosted by the processor 104 and using the communication links 120.

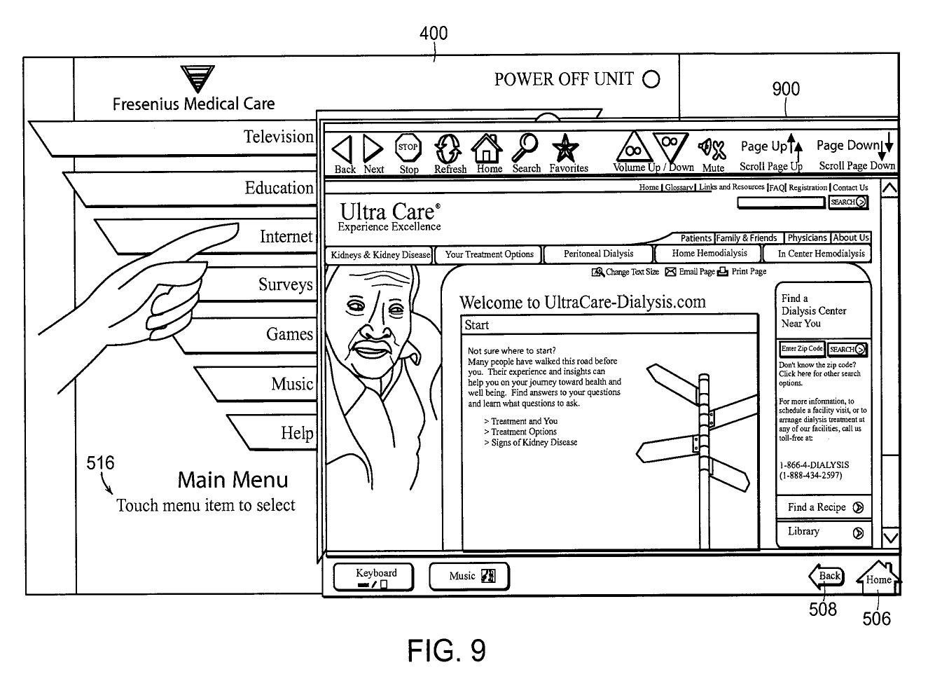

Referring to FIG. 9, if the internet option 406, 406a is chosen (FIG. 4), then the touch screen 102a, 102b can display an Internet screen 900. The Internet screen 900 is shown as a separate, popup window from the menu screen 400 from which it was selected. The Internet screen 900 can function like any browser, as is well known in the art. The Internet screen 900 can provide access to any one or more networks, such as the Internet, an intranet, an extranet, and any other similar type of content-providing network. The network(s) available through the Internet screen 900 can be direct-connected and/or networked to the processor 104 and/or interface 102a, 102b.

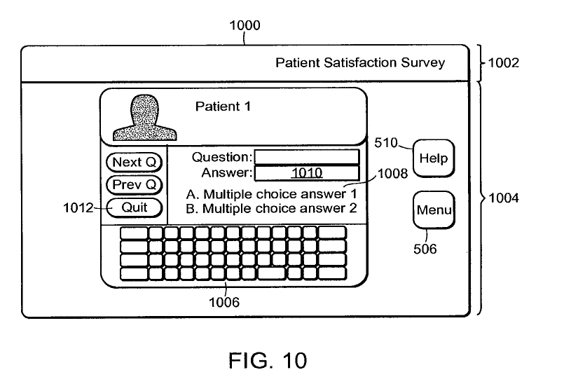

Referring to FIG. 10, if the surveys option 408, 408a is chosen (FIG. 4), then the touch screen 102a, 102b can display a surveys screen 1000. The surveys screen 1000 illustrates a survey in progress. Before displaying the surveys screen 1000, the touch screen 102a, 102b may first display one or more choices of surveys that the patient 112a, 112b can choose to begin (new surveys) and/or complete (in-progress surveys). Surveys can include any question/answer type interaction, such as a feedback form, a questionnaire, or other interrogatory process regarding any aspect of patient care such as how the patient 112a, 112b is feeling, the patient's response to treatment, the patient's understanding of his/her treatment and condition program, the patient's understanding of what he/she should be doing in connection with his/her treatment program, functionality of the station 100a, 100b and other similar types of questioning.

In this embodiment, the surveys screen 1000 includes a title bar 1002 indicating the content being viewed and a content window 1004 showing the actual content. The screens displayed to a particular patient typically have the same general configuration (color scheme, font, button locations, etc.), e.g., the surveys screen 1000 having the same configuration as the music screen 700 (unlike in these illustrated examples where the surveys screen 1000 and the music screen 700 have different configurations). The surveys screen 1000 includes a keyboard 1006 which the patient 112a, 112b can use to enter one of the provided choices 1008 in an answer box 1010. Alternatively, the user may be able to touch one of the provided choices 1008 by way of answer. One or more survey navigation buttons 1012 can be provided which can allow the patient 112a, 112b to, for example, return to the previous question, move to the next question, or quit the survey.

Operation

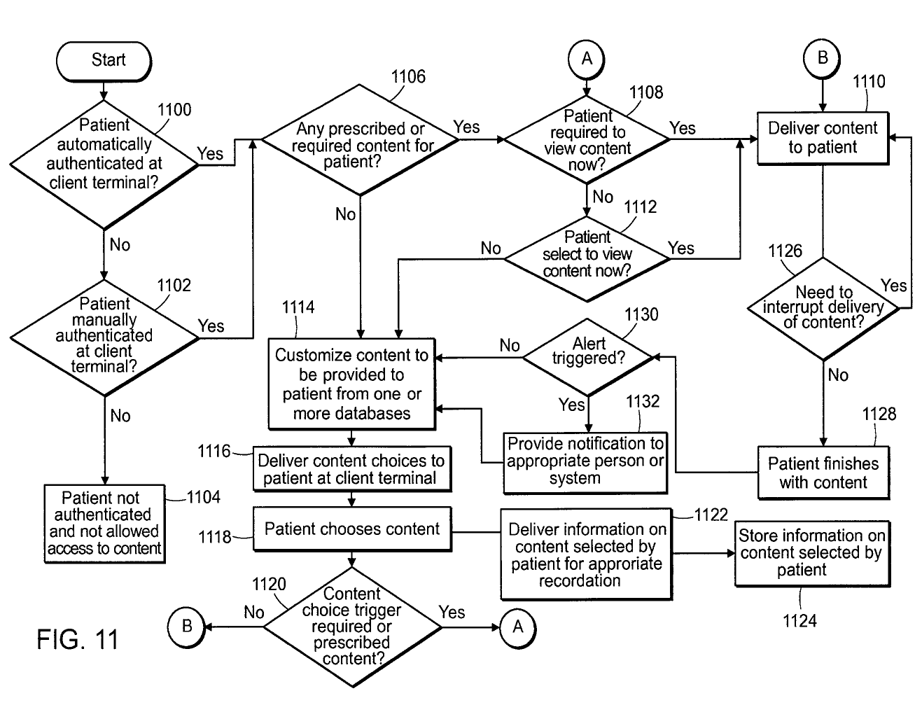

FIG. 11 is a flow diagram illustrating operation of systems according to the invention. Although the system flow diagram is described with reference to the elements included in the embodiments described above, this or a similar process, including the same, more, or fewer steps, reorganized or not, may be performed in connection with other embodiments of the invention. Furthermore, for simplicity of discussion, the system flow diagram is described with reference to the treatment station 100a, although the same can apply to treatment station 100b (and any other station as described herein).

As shown in the drawing, the processor 104 determines 1100 if the patient 112a has been automatically authenticated at the treatment station 100a, and more particularly at the display device 102a. Authentication of the patient 112a can allow the processor 104 to provide customized information to the display device 102a based on the patient 112a currently using the display device 102a. Authentication can also help assure that only authorized users can access data, some of which may be confidential medical information, available at the display device 102a via the processor 104.

In instances where the treatment station 100a is located in a clinical setting (e.g., a hospital or a hemodialysis clinic), automatic authentication can occur, for example, if an administrator authenticates the patient 112a by identifying the patient and entering authorization clearance at the touch screen 122. (In the illustrated embodiment, authentication is not required in instances where the treatment station 100a is disposed in a nonclinical setting (e.g., in the patient's home), though, other embodiments may vary in this regard.

In another example, the patient 112a may be wearing or carrying an automatic authorization mechanism such as a radio frequency identification (RFID) tag that the display device 102a and/or the processor 104 can automatically detect when the patient 112a is within sufficient proximity. In instances where the treatment station 100a is located in a clinical setting (e.g., a hospital or a hemodialysis clinic), the touch screen 122 can be utilized in connection with RFID-based authorization instead or in addition to the display 102a and/or processor 104.

In yet another example, the machine 110a may be programmed for the patient 112a, and the processor 104 can identify the patient based on the machine's programming.

If the patient 112a has not been automatically authenticated, then the processor 104 determines 1102 if the patient 112a can be manually authenticated. Manual authentication can occur using the entry screen 300 described above. If a first attempt at authentication fails, the patient 112a may receive one or more additional attempts to log on to the system, although the number of additional attempts may be limited. If the patient 112a cannot be manually authenticated, the processor 104 denies 1104 the patient 112a any further access to content through the display screen 102a. The processor 104 can also optionally trigger an error message or alert to be sent to an administrator, or more specifically to the touch screen 122 or other device (e.g., touch screen monitor 1324B coupled to remote digital data processing system 1324), so an administrator can assist the patient 112a with login.

Once authenticated either automatically or manually, the processor determines 1106 if there is any required or prescribed content to be displayed to the patient 112a on the touch screen 102a. Required or prescribed content can include any content that has been recommended or prescribed (typically by a physician or other health care provider) for the patient 112a to view, either at the time it is presented or at a later time, typically during the same treatment session in which it was first presented to the patient 112a. The required or prescribed content can be specific to the patient 112a (e.g., regarding a recently prescribed medication or an aspect of treatment) or it can be general (e.g., how the machine 110a works). The processor 104 may consider the patient's location in determining whether required or prescribed content exists. For example, patients who are receiving treatment at a treatment station 100a located in a clinical setting (e.g., a hospital or a hemodialysis clinic)—e.g., where they can ask follow-up questions directly to a heath care provider—may receive different documentation than than those who are receiving treatment at a station 100a that is located in nonclinical setting (e.g., in the patient's home).

By way of non-limiting example, required or prescribed content can include educational materials regarding treatment prescribed by medical personnel (e.g., a physician, social worker, dietician, etc.), instructions regarding the patient treatment station 100a, question of the day, tip of the day, appointment reminders or recommendations, and other types of similar content. That content may be provided in a more abbreviated document, for example, when delivered to a patient who is receiving receiving treatment in a hemodialysis clinic, e.g., since follow-up questions can be answered directly by the nurse or other provider. On the other hand, that same content may be delivered in a more complete form, when delivered to a patient who is receiving treatment at home.

If the processor 104 determines that there is required or prescribed content for the patient 112a, the processor 104 determines 1108 if the patient 112a must view the required or prescribed content now, i.e., before the patient 112a can choose content for viewing on the touch screen 102a and/or before the touch screen 102a displays content option choices (e.g., the menus screen 400) to the patient 112a. If the patient 112a must view the required or prescribed content at the instant time, then the processor 104 retrieves the required or prescribed content from the appropriate one or ones of the databases 106, 108 and delivers 1110 the required or prescribed content to the touch screen 102a for display to the patient 112a. If the patient 112a need not view the required or prescribed content at the instant time (e.g. because the patient 112a can choose to view the required or prescribed content anytime within a certain amount of time, such as a week), the patient 112a can be presented with the option to view the required or prescribed content.

The processor 104 can determine 1112 whether the patient 112a selects to view the required or prescribed content by, e.g., receiving an input signal from the touch screen 102a. If the patient 112a selects to view the required or prescribed content, then the processor 104 retrieves the required or prescribed content from the appropriate one or ones of the databases 106, 108 and delivers 1110 the required or prescribed content to the touch screen 102a for display to the patient 112a.

If the patient 112a does not select to view the required or prescribed content, or if there was not any required or prescribed content for the patient 112a, then the processor 104 customizes 1114 content to be retrieved from and provided to the patient 112a from one or both of the databases 106, 108 (typically, the content database 108). One skilled in the art will appreciate that the content for delivery to the screen 102a can be chosen with a variety of schemes that extrapolate, average, and/or rank content based on such factors as current patient data, previous patient data, and/or “normal” patient values.

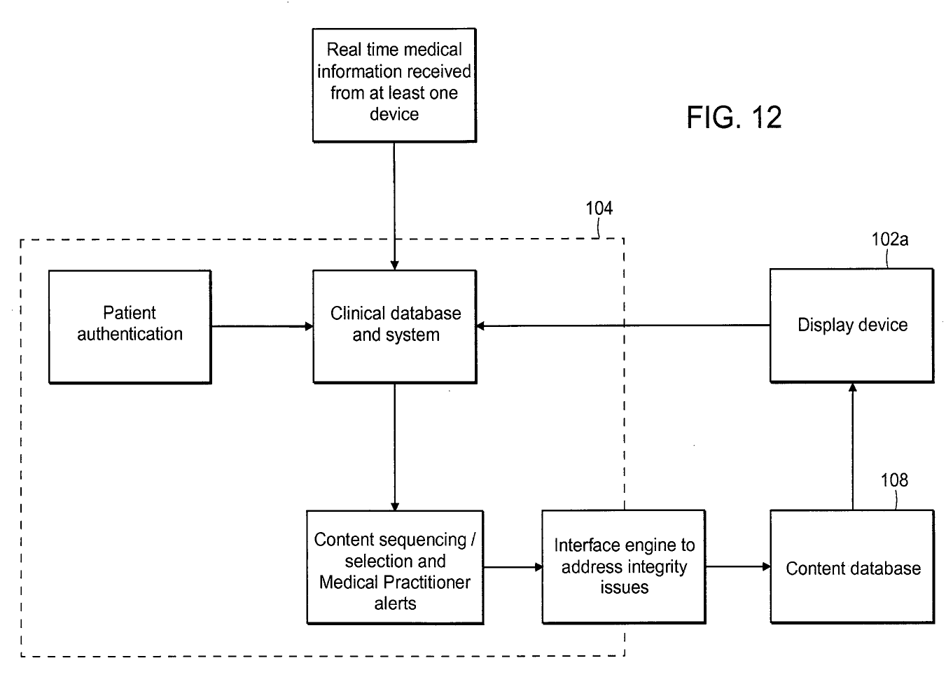

FIG. 12 depicts functional aspects of the processor 104 and elements according to one practice of the invention which can receive real-time data (e.g., from the machine 110a and/or its attendant sensors 1328A-1328D and/or 1330A-1330D, from the screen 102a, etc.), analyze that data to determine content delivery, and deliver content to the touch screen 102a.

Referring back FIG. 11, at the beginning of patient treatment session, i.e., typically immediately following authentication of the patient 112a, the processor 104 may choose content for delivery based on real-time data (e.g., blood pressure, weight, glucose level, hemoglobin level, etc., e.g., from the sensors 1328A-1328D, and/or operational data, e.g., from sensors 1330A-1330D), as well as on non-realtime and/or historical data. For example, the processor 104 can consider archived patient medical data (such as that stored in the medical database 106) indicating diagnosed diseases, previous diet instructions, currently prescribed medications, composition of used or spent dialysate, and the like, and deliver specifically related educational information.

For another example, the processor 104 can consider the patient's current medical condition (e.g., as measured by sensors 1328A-1328D) and/or the patient's compliance with one or more requirements, goals, or milestones (e.g., as determined dosing, on/off times, etc., determined by modules 1332 from operational sensors 1330A-1330D) in determining content choices to present on the display screen 102a. Whether the patient 112a has complied with a requirement, met a goal, or met a milestone can be stored in the database 106 (or other location accessible by the processor 104), and the processor 104 can grant or deny the patient 112a access to particular content from the content database based on the patient's compliance.

For example, after viewing a certain type or amount of educational and/or required material, a new entertainment option such as the Internet can be made an available mode choice as a “reward” to the patient 112a. For another example, if the patient 112a has met a treatment-related goal, such as appearing for all scheduled hemodialysis appointments within a certain time period (e.g., one month) or demonstrating a reduced potassium level over time, then the patient 112a may be granted a reward such as free access to otherwise fee-based content such as cable television or coupons to stores (which may be electronically redeemed using the screen 102a or provided in hard copy to the patient 112a). For yet another example, if the patient 112a previously failed to comply with a requirement or failed to meet a goal, then the processor 104 may deny the patient 112a access to particular content, e.g., games, premium cable television channels, etc. Access to denied content may be restored in real-time, such as if the patient 112a views an educational video on the screen 102a or, in instances where the patient is receiving treatment in a clinical setting, after the patients discusses a missed goal with a medical practitioner who may manually restore patient content access via the screen 122.

Once the processor 104 has determined content for the patient 112a, the processor 104 can deliver 1116 the content to the patient 112a (or, more specifically, the touch screen 102a). This delivered content here generally includes choices of content (video, audio, games, etc.) that can be delivered from the content database 108 upon selection by the patient 112a, such as a customized menu of options on the main menu screen 400 or customized educational materials available through the education screen 600.

Presented with two or more content options, the patient 112a can choose 1118 via the touch screen 102a content he/she wishes to access. The patient's choice can be communicated to the processor 104, which can determine 1120 whether the patient's content choice triggers delivery of required or prescribed content. The processor 104 can also optionally deliver 1122 information identifying the content selected by the patient 112a to an appropriate location for appropriate recordation 1124 (e.g., to one or both of the databases 106, 108 where it may be later accessed by the processor 104 to determine content for delivery to the patient 112a, or even to other patients since the processor 104 may choose content based on popularity among patients having similar medical conditions). If no required or prescribed content is associated with the patient's choice, then the processor 104 can deliver 1110 the selected content to the display screen 102a.

Delivery of required or prescribed content may be triggered for a variety of reasons, such as requiring the patient 112a to view instructions for a game or audio player before being allowed to access it or requiring a patient to watch part one of an educational video before allowing them to watch a subsequent part of the video. If delivery of required or prescribed content is triggered by the patient's selection, the required or prescribed content can be delivered to the touch screen 102a as described above (including not necessarily providing to the patient 112a at the instant time).

Customization of content for delivery 1110 can be an ongoing process for the processor 104. For example, the processor 104 can receive real-time medical information from the machine 110a, e.g., from the sensors 1328A-1328D and/or sensors 1330A-1330D (and/or other device, e.g., a standalone blood pressure monitor, weight scale, glucose meter, home hemoglobin analyzer, or supplemental oxygen device), regarding a current medical condition of the patient 112a. Non-limiting examples of real time medical information include blood pressure, heart rate, blood potassium level, content analysis of fluids transferring in and out of the patient 112a during dialysis treatment, weight, glucose level, hemoglobin level, and other types of similar information that may be monitored and/or catalogued regarding the patient 112a. Real time medical information can be communicated from machine 110a to the processor 104 for storage in the medical database 106 where it can be accessed at a later time, possibly for analysis regarding medical trend information that may be used by the processor 104 to determine what content to deliver to the patient 112a during the instant or subsequent treatment.

Considering real-time medical information and/or other factors such as a duration of the instant treatment session (e.g., as determined from sensors 1330A-1330D) and a history of Internet websites visited, the processor 104 can determine 1126 if there is any need to interrupt delivery of content to the patient 112a. Interruption may be necessary for a variety of reasons. For example, real time medical information regarding the patient 112a received at the processor 104 may trigger delivery of content, e.g., a blood pressure measurement above a certain predetermined level (generally or specific to the patient 112a) may trigger a delivery of a message to the patient 112a that a high blood pressure level has been detected and that they should attempt corrective action such as reclining. In another example, the machine 110a may begin a new stage of treatment or be nearing end of treatment, and the processor 104 can send an alert to the patient 112a informing him/her of the treatment's progress. In yet another example, a high potassium level measurement may trigger delivery of dietary information on how to reduce potassium levels in the blood, including lists of foods to avoid eating and cooking recipes.

If interruption of content delivery to the touch screen 102a is necessary, then the processor 104 can deliver 1110 the newly determined content to the patient 112a. The content viewed by the patient 112a at the time of interruption may be paused for continuation at a later time. If there is no need for interruption, the patient 112a can finish 1128 viewing the content, e.g., finish playing a game, finish watching a video, listen to music until the patient 112a stops it, read educational pamphlet material, browse the Internet until the patient 112a closes the browser, etc.

In some embodiments of the invention, the processor controls the selection of delivered content in accord with the following guidelines:
Can be mandatory videos, such as one on potassium levels.
Survey may trigger other required or recommended content
Can periodically (for example, every hour) require additional content to be received/viewed.
Appointments with dietitians or other healthcare providers can be recommended or required.
Educational games such as constructing a nutritionally appropriate meal should/could be included; can play against others.
Can be based on information from tracking Internet searches, TV viewing, or other content or activities of the patient. For example, by tracking web surfing and/or chatroom activity it may be determined that the patient is seeking information on treating headaches and information on headaches could be provided or required.

After the patient 112a finishes 1128 viewing particular content, the processor 104 can customize 1114 a new set of content (e.g., menu options) for delivery to the patient 112a, or the processor 104 can provide previously determined content. However, based on the content the patient 112a finished, an alert may be triggered, so the processor 104 determines 1130 if an alert is triggered and, if so, provides 1132 notification of the alert to an appropriate person or system.

Alerts can be triggered for a variety of reasons. For example, they may be reported to processor 104 directly by components of station 100 and/or apparatus 110. Alternatively, or in addition, they can be triggered by the monitoring logic 1332 detects that readings from the physiometric sensors 1328 and/or operational sensors 1330 fall outside of selected ranges. That monitoring logic can also, by way of non-limiting example, monitor when and for how long the various sensor readings, for example, are outside those ranges.

For another example, the patient 112a may have completed a survey, and an alert may be generated and sent by processor 104 to inform an administrator, e.g., working locally to treatment station 100a and/or working at the remote system 1324, to forward the survey results to an appropriate survey coordinator. For still another example, particularly if the patient 112a is under a certain age, finishing a game with a score above a certain level may trigger an alert by the processor 104 to be sent (e.g., via electronic mail, text message, phone, pager, illuminating a light proximate to the display screen 102a and/or (in instances where the patient is receiving treatment in a clinical setting, the touch screen 122, etc.) to such an administrator, e.g., in the treatment room, who can deliver a prize to the patient 112a. For yet another example, if the patient 112a views a particular educational video, an alert may be sent by the processor 104 to such an administrator, e.g., in the treatment room, to provide the patient 112a with particular materials (e.g., further reading, materials useful in implementing a taught procedure, etc.) when the patient 112a finishes treatment at the machine 110a. In each of these cases, alerts triggered by processor 104 can be output to screens 102a and/or 112, instead of or in addition to being sent via electronic mail, text message, etc.

In addition to generating and reporting alerts locally, e.g., by processor 104, such alerts can be generated by remote system 1324, e.g., based on like data transmitted to it by processor 104. For this and other purposes, the communications logic (e.g., network interface 1320) operates under control of the processor 104 to transmits treatment data, e.g., query/survey responses to the digital data processing system 1324, along with readings from sensors 1328A-1328D and 1330A-1330D, video (or still images) from the web camera coupled to the touch screen 102a, 102b, and alerts from monitoring logic 1332, etc., to system 1324 for consideration by health care provider 1322 and/or reporting/analysis by module 1324 in accord with the teachings hereof. In the illustrated embodiment, that information is transmitted e.g., at or around the time of completion of each treatment session, though, they can be transmitted at regular intervals (e.g., daily, weekly and so forth), e.g., when the device 110a, network 28 and/or data processing system 1324 is otherwise amenable to such transfer.

As noted and in addition to the above, features of systems according to the invention may include a capability to track the inventory and/or use of supplies (including without limitation erythropoietin, PD solutions, other medicines, and other supplies) in the patient's home, e.g., as determined from readings taken from the operational sensors 1330A-1330D, from querying the patient, or otherwise. Such data can be compared by processor 104 against measures or estimates of existing supply and/or prior history of supply use, and changes, increases or decreases can be noted and charted or graphed, e.g., on screens 102a and/or 112. Software executing on the processor 104 can, further, analyze the data and/or results of those comparisons, e.g., using expert systems logic, operational analysis techniques, statistics, or the like, all of the type known in the art, as adapted in according the teachings hereof, e.g., to reach conclusions regarding underlying causes and/or resulting conditions, which can also be displayed on screen 102b. In addition to, or in lieu of, of the foregoing, the processor can also signal alerts pertaining to supply use, changes therein, underlying causes and/or resulting conditions.

Such conclusions might include, by way of illustrative example, concluding that their low supplies at the patient's home, excessive use of supplies, unusually low use of supplies, use of supplies inconsistent with a care plan, use of supplies in conformance with a care plan, etc.

Alternatively, or in addition, the aforesaid comparisons and analysis can be performed by module 1324A based on data transmitted to system 1324 by processor 104. And, more generally, all of the activities attributed herein to processor 104 may, in some embodiments, be performed by processor 1324 and vice versa.

Regardless, following such analysis(ies) performed by processor 104 and/or system 1324, the patient, care provider(s), insurer, third party, etc., can be notified via electronic mail, text message, phone, pager, or otherwise, of the inventory and/or use of supplies, related charts or graphs, and issues and conclusions that may be derived therefrom (low supplies at the patient's home, excessive use of supplies, unusually low use of supplies, use of supplies inconsistent with a care plan, use of supplies in conformance with a care plan, etc.).

Among other things, if the inventory at the home is low, the processor 104 and/or system 1324 may trigger a resupply from a care provider, third-party, etc., e.g., by automatically effecting the shipment of supplies to the patient and/or or by generating a notice to an appropriate person that the product inventory is low and resupply may be required.

Also as noted and in addition to the above, features of systems according to the invention may include facilitating analysis of patient related data at a location different than the patient's home, such as a central office, and communication of patient related data and related analyses to health care providers. For example, patient weight data collected by sensors 1328A-1328D may be transmitted by processor 104 to system 1324, where module 1324A may be chart or graph weight changes over time, showing weight gain or loss trends, and may output such chart/graph via device 1324B and/or signal the patient, care provider(s), insurer, third party, etc., via electronic mail, text message, phone, pager, or otherwise, of the results. More generally, query/survey responses to the digital data processing system 1324, along with readings from sensors 1328A-1328D and 1330A-1330D, and alerts from monitoring logic 1332, may be transmitted by processor 104 to system 1324 for reporting and/analysis consistent with the teachings hereof—including provision of content, generation of alerts, and/or performing other functions attributed elsewhere herein to processor 104.

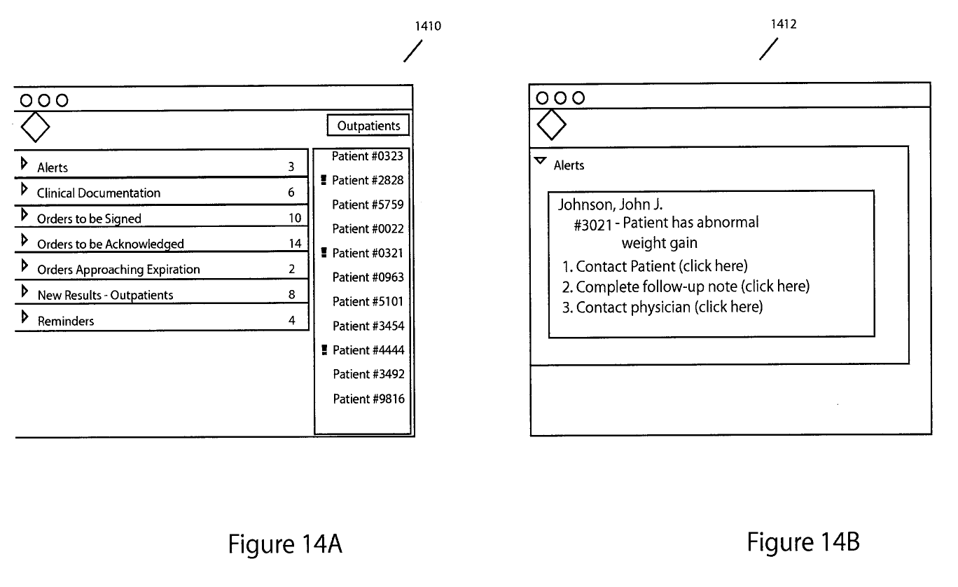

FIG. 14A depicts a screen 1410 of the type generated by module 1324A for display on touch screen 1324B identifying a list of active patients and a worklist (or workflow) of actions to be taken by a nurse or other health care provider with respect to them. Included in the display are exclamation points (or other indications) of patients for which alerts have been generated, e.g., at the patient treatment station and/or by module 1324A: here, three such alerts are indicated.

When a nurse or other health care provider selects the “Alerts” drop-down on the touch screen (or alternatively clicks on the exclamation point adjacent the patient's I.D.), the system 1324 generates a screen 1412 of the type shown in FIG. 14B identifying the alert condition for the specific patient. Here, that alert concerns abnormal weight gain. The module 1324A also generates on that screen a workflow of tasks for the nurse to execute in response to the alert, e.g., contacting the patient, making a note of the contact in the patient's record and contacting the physician. In the illustrated system, the nurse can select those items on the touch screen in order to initiate the respective task, e.g., automatic phone dialing, pulling up of a ‘contact note’ dialog box, etc.

Although shown in FIGS. 14A-14B and discussed above as being generated by module 1324A on touch screen 1324B, like screens can be generated by processor 104 for display on touch screen 122 and/or for transmission to, and display on, touch screen 1324B.

A further appreciation of operation of systems according to the invention may be attained by reference to the following script for a video depicting use and operation of systems according to the invention at a treatment center:

Narrator:
Welcome to the Clinic. We'll see how our new clinical system [constructed and operated in accord with the teachings hereof] supports patient care management and clinical decision making.
We'll also see how [touch screen 122] frees up the clinic staff to spend more time with the patient.
Finally, we'll see how our patients use the new Direct-Touch Patient Entertainment System, as we deliver on the
UltraCare promises of Individual Patient care and excellent customer service.
(In the video, the nurses have are reading and/or entering data via touch screen 112. The patient, Gretchen, is seated taking treatment from apparatus 110a and interacting with touch screen 102a)
[The nurses have] been taking care of Gretchen, but let's see what she's been so busy doing!
With the patient treatment station [constructed and operated in accord with the teachings hereof], Gretchen has access to digital radio, satellite TV, movies, games, educational material, and the internet.
Gretchen, do you mind showing us the DirectTouch system?

Gretchen:
(Gesturing to touch screen 102a) Hi! Let me show you how I use this! It's a touch screen so I just pick exactly what I want from the menu.
The best thing about this system is that I get to watch what I feel like—sometimes it's a movie, and sometimes it's a video to help me understand how to eat right . . . .
My dietitian says I'm doing a much better job these days managing my Po-tah-see-um. I think it's really helped to review the rules right here whenever I want to. And here's something I didn't expect—satellite radio!
(Touching screen 102a and pretending to sing) OH! I love this song! (She listens to the music for a few seconds).
I don't suppose we have time to listen to the whole thing . . . it reminds me of a handsome Swedish gentleman I met once.
(Glancing at screen 102a) Let's see—where next? Surveys? That's where I can let the staff know how things are going, and so on.
(Glancing toward video monitor/audience and giving thumbs up) But let me show you the games—this really helps when the treatment seems long . . .
There's card games and also board games—I'm getting pretty good!
And I can even access the internet! It's neat, and I guess I'm going to use this a lot in the future.
I think I've covered it all. Now I think I'll get back to my movie if you don't mind! Thanks for stopping by!

A still further appreciation of operation of systems according to the invention may be attained by reference to the following script for a video depicting use and operation of systems according to the invention in instances where the patient and patient care station (namely apparatus 110 and processor 104) are located at the patient's home, whereas the health care providers are disposed remotely (e.g., at the hospital or central treatment center) with access to digital data processing system 1324:

Narrator:
(Video image of architectural blueprint of patient's home transitions to “zoom/fly in” to patient's bedroom, with patient seated on bed, near dialysis machine 110, touch screen 102a and processor 104.) [System according to the invention improve] patient awareness and compliance while enhancing the self-care process and facilitates physician interaction.

(Video images zooms in to touch screen 102a. Video image transition to screen with menu and examples, e.g., as shown in FIG. 4 hereof). Home care patients can use the intuitive touch-screen interface to choose from a variety of activities and programs. They can view educational materials and messages targeted to their specific needs. They can also access lab results, personalized nutrition advice, surveys and even a patient report card.
(Video depicts animated graphic showing data from apparatus 110a and other diagnostics in patient's home “flowing” into computer 104.) Because [the touch screen 102a and processor 104 are] connected directly to the [treatment apparatus 110a], at home, all pertinent data and vital signs flow straight into the system. The system can connect to other diagnostic instruments [in the patient's home], as well. Devices include scales, blood pressure cuffs, glucose meters and many more . . .
(Video shows dialysate and other supplies for apparatus 110a appearing on graphic and then cuts to video of real supplies in patient's home.) The system even tracks inventory data for each patient so supplies and deliveries can be adjusted more efficiently, according to individual needs.
(Video shows screen shots of touch screen of remote digital data processing system 1324 with graph of data transmitted from processor 104 and/or analyses thereof generated by module 1324A). Sophisticated embedded analytics put metrics and critical information in the hands of clinicians, instantly.
(Video shows patient at home getting on weight scale! A graphical animation shows data from that scale traveling to processor 104 and, from there, to digital data processing system 1324). With the system's technology, clinicians will have a better and more current view into their patient's treatment. For example, the system can record the patient's vital signs & weight, without the need for a paper log.
(Video shows nurse viewing touch screen of remote digital data processing system 1324. On screen is a worklist of alerts, one of which is the alert “Unexpected Weight Gain.” Nurse clicks on that alert, using digital data processing system 1324). The system's workflow engine [e.g., executing on processor 104 and/or module 1324A of computer 1324] which is always processing information, identifies the weight gain trend. It notifies the home support nurse, so the next time she reviews her work list, she can view the details with a click.
(Video shows touch screen of remote digital data processing system 1324 with trended graph of patient's weight and blood pressure, showing unexpected weight gain.) A trend graph confirms the unexpected weight gain.
(Video shows nurse using digital data processing system 1324 to pull up on touch screen of that system a clinical summary of the patient's history). She can then access & review the patient's full record, with another click of the mouse, and determine whether contacting the patient is necessary.
(Video shows nurse using digital data processing system 1324 to click on information displayed on the touch screen of that system. The screen displays “Dialing Mr. Johnson”.)

Nurse:
“How are you Mr Johnson? I'm calling because I noticed that you've been gaining weight . . . ”

Narrator:
(Video shows nurse talking to patient; she, located at the treatment center; he, at home). The system's technology gives patients a digital lifeline so they don't have to feel isolated and alone, simply because home dialysis is their best option.
(Video shows nurse hanging up from phone call. Using digital data processing system 1324 she pulls up on the monitor of that system a “Progress Note” screen to document the call. Video shows that screen with some data preentered/template) After the call, it's easy to enter a progress note . . . and convenient templates ensure that the standard work flow is followed.
(Video shows screen displayed on touch screen of digital data processing system 1324. Bottom of screen shows order note from physician asking to be alerted on weight gain. Nurse clicks on “Yes” button on screen using system 1324.) When the physician requests it, the nurse can create an alert on the physician's worklist so he will be aware of any unusual trends.
(Video shows overhead shot of physician's office.) The physician can then review the alert . . . and access the full medical record if necessary.
(Video shows touch screen of another computer of digital data processing system 1324 displaying physician's work list, including the alert of the patient's unexpected weight gain.) He can also choose to change the order . . . or leave a note for the nurse . . . or simply acknowledge the alert.
(Video shows physician using that computer to pull up from digital data processing system 1324 the patient's clinical summary). With the system, physicians, as well as clinical staff, dietitians and social workers can access all of their patients' data from remote locations, and then interact with one another and the patient, at any time. In effect, it's like making a house call—online.

Physician:
(Video shows touch screen 102a at patient's home displaying live video image of physician, while he and patient engage in video “chat”) Hi Mr. Johnson. I wanted to talk to you because I see that you had a question, yesterday, about your access . . .

Narrator:
(Video continues to show touch screen 102a as patient at home continues to talk to physician on screen). The system brings clinic levels of support to the physician and home therapy staff, increasing visibility to the patient, and enabling better individualized care for every patient, every day, every home.
(Video shows blueprint of patient's home). With the system, physicians and home therapy staff have the tools to support home patients as if they were in-center, enabling better individualized care for every patient, every day, in every home. For patients, this means a greater sense of safety, efficiency and confidence when they dialyze at home.

Described above are devices, methods and systems meeting the aforementioned objects, among others. Benefits of such devices, methods and systems include:
Patients more likely to take treatments as prescribed and complete treatments.
Provides alternate focus from pain; makes treatment a less painful experience
Improves patient compliance with treatments
Improves therapeutic outcomes
Improves patient care results may increase physician compensation/reimbursement
Improves patient interactivity/communication with healthcare provider
Enhance patient self-care
Enhance provider responsiveness
In regard to monitoring and/or provision of supplies to the patent, advantages include: tailoring delivery process for patient (automate re-ordering. trigger call from customer service); Inventory management (scan supply barcodes; lower inventory.); track usage of dialysate solutions, EPO and other medicines and/or supplies; facilitate notification to patient, care provider, insurer, etc.; trigger resupply from care provider, third-party, etc.
Physician will have more access to patient (decrease sense of “out-of-sight, out-of-mind”; improved ability to manage patient)
Decrease drop-out rate by better support to patient
Improved Patient training/education/support
Home Nurses, Dietician, & Social Worker can focus on exceptions, as driven by workflow/alerts
Overall: increases value of treatment to patients, their families, and clinicians/physicians.
Connectivity & communications
   Patient data available in near-real-time
      Integrate into clinical workflows, alerts
      Improved information flow to clinicians/physicians
      Increase billing frequency for commercial payers
   Patient reminders (EPO (Erythropoietin) days, etc)
   Patient could get additional support/training over the network as needed (ask questions)—deal with patient concerns/fears
   Web camera
      physician/clinician can see patient
      Equipment troubleshooting.
Better data collection for use by care providers (including dietician and social worker to insure compliance) and for home medicine management.
Simplicity for patient
Small enough that patient can keep it where it is convenient
Equipment in patient's home can be battery powered or powered by wall outlet and can be embodied in different form factors
   PDA
   All-in-One PC
   Laptop
Can take advantage of bandwidth/connectivity trade-offs.
Avoids unnecessary action by patient (including avoiding requirement of asking patient to track treatment record, bring same to clinic, etc.)
Avoids requiring physician/clinician to needlessly acquire and analyze information in patient's home treatment regime (use workflows-worklists/Use Alerts; Focus on Problem Resolution; Information is transmitted automatically to central database).
Home vs Clinic-Based use of patient treatment station:
   Users & clinician not competing for access to hardware
   Only have to set up one network connection/session
   Same authentication process covers usage for charting & entertainment/education
   CCPD & home-HD patients are sleeping while cycling
   Patients are at home—already have access to their choice of entertainment (TV, DVD, radio, books, etc)
   Deliver patient report cards, tailored education

Those skilled in the art will appreciate that the embodiments discussed and shown herein are merely examples of the invention and that other embodiments fall within the scope thereof. Thus, by way of non-limiting example, it will be appreciated that a patient treatment station of the type described above can be used with a variety of medical treatment and diagnostic apparatus, in addition to dialysis machines. By way of further example, it will be appreciated that, in some embodiments (e.g., where sanitary conditions are of less concern and/or are compensated for in other ways), the touch screens can be replaced by a conventional displays (e.g., cathode ray tube (CRT), liquid crystal display (LCD), or otherwise) and a keyboards or other input devices. By way of still further example, methods, systems and devices according to the invention may utilize lesser, more and/or other elements than those shown in the drawings and described herein, and that the functionality of various ones of the elements shown or describe here may be incorporated into and/or otherwise combined with other elements.

In view thereof, what we claim is:

1. A method of patient care, comprising:
detecting a current medical condition of a patient receiving medical treatment, wherein the detecting step includes any of sensing at least one of blood pressure, heart rate, weight, glucose level, hemoglobin level, and blood potassium level, and
transmitting information regarding the medical condition to a digital data processing system disposed remotely from the medical treatment apparatus.

2. The method of claim 1, wherein the medical treatment apparatus includes one or more sensors utilized in connection with peritoneal dialysis and hemodialysis treatment.

3. The method of claim 1, further comprising delivering to a health care provider from the remote digital data processing system up-to-date medical condition data.

4. The method of claim 1, wherein the delivering step includes generating trend graphs with the medical condition data.

5. The method of claim 1, further comprising
acquiring any of a still or video image of the patient, and
transmitting that image to the remotely-disposed digital data processing system.

6. A method of patient care, comprising:
detecting a medical condition of a patient receiving medical treatment, wherein the detecting step includes any of receiving data from a medical treatment apparatus coupled to the patient,
accepting from the patient a subjective characterization of any of medical condition and/or medical treatment,
transmitting information regarding any of the medical condition and the subjective characterization to a digital data processing system disposed remotely from the medical treatment apparatus.

7. The method of claim 6, wherein
the step of receiving data from the medical treatment apparatus includes receiving data from one or more physiometric sensors that take biometric readings of the patient, and
the step of transmitting information to the remotely-disposed digital data processing system includes delivering data from one or more of those physiometric sensors to that digital data processing system.

8. (canceled)

9. The method of claim 6, wherein
the step of receiving data from the medical treatment apparatus includes receiving data from one or more operational sensors that sense operating conditions of the medical apparatus, and
the step of transmitting information to the remotely-disposed digital data processing system includes delivering data from one or more of those operational sensors to that digital data processing system.

10. (canceled)

11. The method of claim 6, wherein
the step of receiving data from the medical treatment apparatus includes receiving image data from a camera coupled to the medical treatment apparatus, and
the step of transmitting information to the remotely-disposed digital data processing system includes delivering data from that web camera to that digital data processing system.

12. The method of claim 6, wherein the medical treatment apparatus is any of a device for peritoneal dialysis and hemodialysis treatment.

13. The method of claim 6, further comprising generating graphs of one or more datum received from the medical treatment apparatus.

14. The method of claim 6, further comprising any of reporting and/or analyzing one or more datum received from the medical treatment apparatus.

15. The method of claim 14, wherein the steps of reporting and/or analyzing are executed by a processor local to the medical treatment apparatus.

16. A method of patient core, comprising:
treating a patient with a medical treatment apparatus,
detecting one or more operating conditions of the medical treatment apparatus via sensors coupled thereto,
analyzing data reflecting those operating conditions to any of track inventory and/or use of supplies in connection with treating the patient.

17. The method of claim 16, where the supplies includes any of erythropoietin, dialysis solutions, other medicines, and other supplies utilized in connection with such treatment.

18. The method of claim 16, comprising responding to the analyzing step by effecting any or ordering and delivery of supplies for use in connection with treating the patient.

19. The method of claim 16, wherein the detecting step includes taking readings from one or more operational sensors coupled to the medical treatment apparatus.

20. The method of claim 19, wherein the step of taking readings includes taking readings of levels or dosings of consumables utilized in connection with treatment.

21. The method of claim 20, wherein step of taking readings of levels or dosing includes taking readings of any of fresh and spent dialysate and supplemental medications.

22. The method of claim 16, wherein the analyzing step includes comparing data reflecting operating conditions of the medical treatment apparatus against any of measures or estimates of existing supply and/or prior history of supply use.

23. The method of claim 22, wherein the analyzing step includes any of analyzing the data reflecting the operating conditions and/or results of said comparisons to reach determine underlying causes and/or resulting conditions.

24. A method of patient care, comprising:
treating a patient with a medical treatment apparatus,
detecting any of an operating conditions of the medical treatment apparatus and a current medical condition of the patient receiving medical treatment,
generating an alert based on one or more of said operating conditions and medical conditions,
displaying such alert, along with a workflow of actions to be taken in response thereto by a health care provider.

25. The method of claim 24, comprising to responding to selection of a displayed action by initiating that action.

26. The method of claim 25, where the actions include contacting a patient, contacting a health care provider and making a notation in a patient record.

27. The method of claim 24, wherein any of the generating step and the displaying step are executed remotely from the medical treatment apparatus.

28. The method of claim 27, comprising transmitting more operating conditions of the medical treatment apparatus to a digital data processing system disposed remotely from the medical treatment apparatus.

29. A patient care system, comprising:
A. a medical treatment apparatus,
B. one or more sensors coupled to the medical treatment apparatus that detect a current medical condition of a patient receiving medical treatment, the medical condition including any of a blood pressure, heart rate, weight, glucose level, hemoglobin level, and blood potassium level, and
C. a processor local to the medical treatment apparatus that transmits information regarding the medical condition to a digital data processing system disposed remotely from the medical treatment apparatus.

30-44. (canceled)

45. A patient care system, comprising:
A. a medical treatment apparatus,
B. one or more sensors coupled to the medical treatment apparatus that detect a current medical condition of a patient receiving medical treatment,
C. one or more sensors coupled to the medical treatment apparatus that detect an operating condition thereof, and
D. any of a touch screen or other output device,
E. a processor coupled to the one or more sensors and to the touch screen or other output device, the processor generating an alert based on the conditions detected thereby and causing that alert to be displayed on the touch screen or other output device, along with a workflow of actions to be taken in response thereto by a health care provider.

46-48. (canceled)
